(19) United States
(12) Patent Application Publication
(10) Pub. No.: US 2018/0264191 A1
(43) Pub. Date: Sep. 20, 2018

(54) SYSTEMS AND METHODS FOR NEURAL DRUG DELIVERY AND MODULATION OF BRAIN ACTIVITY

(71) Applicant: Massachussetts Institute of Technology, Cambridge, MA (US)

(72) Inventors: Canan Dagdeviren, Cambridge, MA (US); Robert Langer, Newton, MA (US); Michael J. Cima, Winchester, MA (US)

(73) Assignee: Massachusetts Institute of Technology, Cambridge, MA (US)

(21) Appl. No.: 15/919,822

(22) Filed: Mar. 13, 2018

Related U.S. Application Data
(60) Provisional application No. 62/470,932, filed on Mar. 14, 2017.

(51) Int. Cl. A61M 5/158; A61M 25/00; A61M 5/172; A61N 1/05

(52) U.S. Cl. CPC A61M 5/158; A61M 25/0084; A61M 5/1723; A61N 1/0529; A61M 2202/04; A61M 2205/051; A61M 2205/02; A61M 2207/00; A61M 2230/08; A61M 2205/054; A61M 2210/0693

(57) ABSTRACT

A neural drug delivery system is disclosed. In an embodiment, the system includes two or more microtubes, each having a distal end, a proximal end, and elongate channel body extending therebetween; an electrode having a distal end, a proximal end, and elongate body extending therebetween; an elongate carrying template supporting the microtubes and the electrode in an aligned stack; and an annular needle having a distal end and a proximal end, and housing the carrying template, the microtubes, and the electrode. The system may include at least one pump fluidically connected to the proximal end(s) of one or more of the microtubes. The pump may be configured to deliver a fluid drug on demand through the elongate channel body and out of the distal end of the microtubes.

### CROSS-REFERENCE TO RELATED APPLICATIONS

This application claims priority to U.S. Provisional Patent Application No. 62/470,932, file Mar. 14, 2017, which is incorporated by reference herein in its entirety.

### STATEMENT REGARDING FEDERALLY SPONSORED RESEARCH OR DEVELOPMENT

This invention was made with government support under Grant No. R01 EB016101 awarded by the National Institutes of Health. The government has certain rights in the invention.

### FIELD OF THE DISCLOSURE

The disclosure generally relates to medical devices and more particularly relates to minimally invasive neural drug delivery systems and methods of use thereof, for example, for modulation of brain activity.

### BACKGROUND

Transformative technologies, such as fMRI, deep brain stimulation (DBS), and optogenetics, have allowed the interrogation and manipulation of neural circuitry with increasingly high spatial and temporal resolution and show promise for therapeutic use. An emerging field is now opening to add molecularly-based therapeutic systems that can deliver neurochemicals to modulate neural functions with cell-type specificity, and equally high spatial and temporal targeting.

The brain harbors many potential drug targets. Many of these targets are receptors and related molecules that exist elsewhere in the body. Systemic administration of drugs will also target these peripheral receptors. Moreover, the brain is an exquisitely heterogeneous organ, in which tissue and cell types and functions vary from location to location on scales ranging from sub-millimeter to many centimeters. This heterogeneity can lead to off-target exposure in the brain and to undesired effects of therapeutic agents. These issues have prompted attempts to deliver drugs directly to the brain using physical means such as by the use of catheters. The most common delivery site is the ventricular system, which contains cerebral spinal fluid. This route can provide drug exposure to much of the brain, but penetrance is uneven and severely compromised by distance from the ventricular system. Increasingly, drug delivery strategies are targeting specific regions of the brain.

Physical targeting to small regions of the brain is challenging. The volume of exposed tissue depends on multiple parameters including concentration of the drug in the delivery medium, volume and rate of infusion and elimination rate of the drug. Delivery volumes used to date range from 10 nL to 6 mL. Many key neural circuit nodes have sub-mm³ volumes and cell-specific identities. Thus, small-volume modulation in drug administration is desirable. The modulation of neural circuit dynamics involves fast, acute intervention with controllable on/off dosing to enable prompt interaction with neural network activity. Probes such as those used in convection-enhanced delivery, however, suffer from diffusion and leakage problems even when turned off due to the large fluidic outlet size and holdup volume within the device.

Because existing neural probes with large dimensions can lead to significant gliosis and related deleterious tissue reaction, smaller probes have been developed with microfabrication techniques. These probes, however, have mainly been intra-cortically applied, penetrating only into the most superficial parts of the brain due to the designed device mechanics (i.e., low bending stiffness, small aspect ratio). A major challenge is presented by the need to access deep brain structures, centrally involved in the complex processes underlying behavior, emotion, and homeostasis. Examples of these disorders are Parkinson's disease and disorders related to mood control.

It therefore would be desirable to provide improved delivery systems for minimally invasively targeting neural tissues sites, addressing one or more of the limitations of conventional systems and techniques described above.

### SUMMARY

Some or all of the foregoing needs and/or problems may be addressed with one or more of the embodiments of the systems and methods described herein.

In one aspect, a drug delivery system is provided. In some embodiments, the system includes (i) two or more microtubes, with each of the microtubes having a distal end, a proximal end, and elongate channel body extending therebetween; (ii) an electrode having a distal end, a proximal end, and elongate body extending therebetween; (iii) an elongate carrying template supporting the microtubes and the electrode in an aligned stack; and (iv) an annular needle having a distal end and a proximal end. The annulus of the needle houses the carrying template, the microtubes, and the electrode. The system also includes at least one pump fluidically connected to the proximal end(s) of one or more of the microtubes, wherein the pump is configured to deliver a fluid drug on demand through the elongate channel body and out of the distal end of the one or more microtubes.

Other features and aspects of the disclosure will be apparent or will become apparent to one with skill in the art upon examination of the following figures and the detailed description. All other features and aspects, as well as other system, method, and assembly embodiments, are intended to be included within the description and are intended to be within the scope of the accompanying claims.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIG. 1 schematically depicts a drug delivery system for neural circuit modulation in a patient in accordance with some embodiments described herein.

FIG. 2A depicts a cross-sectional view of a stack of temporary substrate comprising an Si/sacrificial layer, and PMMA/PI in accordance with some embodiments described herein.

FIG. 2B depicts a photoresist layer on the stack in FIG. 2A in accordance with some embodiments described herein.

FIG. 2C depicts a photoresist pattern for etching the underlying layer of PI to define the trench of the PI template in accordance with some embodiments described herein.

FIG. 2D depicts selective etching of the PI layer in accordance with some embodiments described herein.

FIG. 2E depicts removing photoresist in accordance with some embodiments described herein.

FIG. 2F depicts a photoresist pattern for etching the underlying layer of PI to define the PI template in accordance with some embodiments described herein.

FIG. 2G depicts the structure of the PI template in accordance with some embodiments described herein.

FIG. 2H depicts, in a cross-sectional view, the aligning of two microtubes (BS) and a tungsten (W) electrode on the PI template in accordance with some embodiments described herein.

FIG. 2I depicts a perspective view of the structure in FIG. 2H in accordance with some embodiments described herein.

FIG. 2J depicts dissolving the PMMA layer in an acetone bath to retrieve the MiNDS components from the temporary substrate of Si in accordance with some embodiments described herein.

FIG. 3A depicts a schematic illustration of a Hamilton needle in accordance with some embodiments described herein.

FIG. 3B depicts an SEM image of the Hamilton needle with a 30° tip angle in accordance with some embodiments described herein.

FIG. 3C depicts a magnified view of the needle tip in FIG. 3B in accordance with some embodiments described herein.

FIGS. 4A-4C depict SEM images of an unpolished BS aligner tip at various magnifications in accordance with some embodiments described herein.

FIGS. 4D-4F depict SEM images of a polished BS aligner tip at various magnifications in accordance with some embodiments described herein.

FIGS. 5A-5C depict SEM images of a MiNDS with a BS aligner tip having a length of 0.8 mm, 1.5 mm, and 2.0 mm, respectively, in accordance with some embodiments described herein.

FIGS. 6A-6E depict SEM images of a MiNDS, including a W electrode and BS channels at various magnifications in accordance with some embodiments described herein.

FIG. 7A schematically depicts a system with an exploded view of the device components in accordance with some embodiments described herein.

FIG. 7B depicts an L-MiNDS and an S-MiNDS with an electrical connection (tungsten, W electrode) and the fluidic channels (borosilicate, BS) in accordance with some embodiments described herein.

FIGS. 7C and 7D depict SEM images of the tip of L-MiNDS at various magnifications in accordance with some embodiments described herein.

FIGS. 8A-8J depict the electrical characterization of an S-MiNDS and an L-MiNDS at 37° C. in saline in accordance with some embodiments described herein.

FIG. 9 depicts impedance vs time graphs for S-MiNDS and L-MiNDS in accordance with some embodiments described herein.

FIG. 10A depicts the average infusion profiles of three infusion trials through S-MiNDS with flow rates 0.1, 1, and 10 μl/hr, where E.I. represents the end of infusion, and T.V. denotes the theoretical value of the volume infused in accordance with some embodiments described herein.

FIG. 10B depicts normalized intensity vs. position graphs across the bolus, wherein the diameter, w, of the bolus was determined using a 3D ROI, and where the borders were defined as 10% of peak core intensity, I, in accordance with some embodiments described herein.

FIG. 10C depicts normalized ROI sum intensity vs. time profile of identical Cu-64 infusions delivered into an agarose phantom (0.6% by wt.) in accordance with some embodiments described herein.

FIG. 11 depicts average infusion profiles of the iPrecio pump through the L-MiNDS (n=4 infusions per profiles), where E.I and T.V. represent the end of infusion and theoretical value of volume infused, respectively, in accordance with some embodiments described herein.

FIGS. 12A and 12B depict average infusion profiles over time for the iPrecio pumps through the L- and S-MiNDS, respectively, in accordance with some embodiments described herein.

FIG. 12C depicts the average infusion profile for a 20 mins infusion at 6 μl/hr, through both S- and L-MiNDSs, where E.I and T.V. represent the end of infusion and theoretical value of volume infused, respectively, in accordance with some embodiments described herein.

FIGS. 13A and 13B depict plots of volume infused (nl) vs time (min), with FIG. 13A at 1 μl/hr and FIG. 13B at 10 μl/hr in accordance with some embodiments described herein.

FIG. 14 depicts GFAP intensity as a function of distance away from the edge of the stab wound from 8 weeks post-implantation in accordance with some embodiments described herein.

FIG. 15 depicts normalized ROI sum intensity vs. time profile of Cu-64 infusions (30 μCi/μl, 4 mins infusion at 10 μl/hr, 667 nl total volume infused) delivered into an agarose phantom (0.9% by wt.), and in the rat brain through implanted S-MiNDSs using a syringe pump and an iPrecio pump in accordance with some embodiments described herein.

FIGS. 16A-16C depict normalized intensity vs. position curves at 5, 10, 15 and 20 mins for Cu-64 infusions (30 μCi/μl, 667 nl total volume infused at 10 μl/hr) delivered into an agarose phantom using a syringe pump, in rat brain through implanted S-MiNDS using a syringe pump, and an iPrecio pump, respectively, in accordance with some embodiments described herein.

FIG. 17 depicts a plot of length of time to reach maximum bolus value as measured using PET imaging vs. Time Implanted in accordance with some embodiments described herein.

FIG. 18A depicts unit firing rate histograms for 1 min bins in accordance with some embodiments described herein.

FIG. 18B depicts representations of sorted and unsorted action potentials based on peak values in accordance with some embodiments described herein.

FIG. 18C depicts averaged action potentials of a well-isolated unit (single-unit) before and after muscimol infusions in accordance with some embodiments described herein.

FIG. 18D depicts the average number of 180° CW and CCW turns at pre-infusion, post-saline and post-muscimol infusion cases for n=6 in accordance with some embodiments described herein.

FIG. 19 depicts unit rate histograms for 1 min bins in accordance with some embodiments described herein.

FIGS. 20A-20F depict color-tracking maps of a rat during the pre-infusion, post-saline, and post-muscimol infusions and corresponding average number of 180° CW and CCW turns at pre-infusion, post-saline and post-muscimol infusion cases in accordance with some embodiments described herein.

FIG. 21A depicts unit rate histograms for 1 min bins in accordance with some embodiments described herein.

FIG. 21B depicts average waveforms for units binned during each period (pre-infusion baseline, aCSF infusion, and muscimol infusion) with standard deviation in gray shading in accordance with some embodiments described herein.

FIG. 22 depicts average waveforms for units binned during each period (pre-infusion baseline, aCSF infusion, and muscimol infusion) with standard deviation in dashed outlines in FIG. 21B in accordance with some embodiments described herein.

FIG. 23A depicts unit rate histograms for 1 min bins in accordance with some embodiments described herein.

FIG. 23B depicts compiled average waveforms for units binned during each period (pre-infusion baseline, aCSF infusion, muscimol infusion, and 2^{nd} aCSF infusion) with standard deviation in dashed outlines in accordance with some embodiments described herein.

FIG. 23C depicts average waveforms for units binned during each period (pre-infusion baseline, aCSF infusion, and muscimol infusion) with standard deviation in gray shading. Vertical and horizontal bars denote 10 μV and 2 ms, respectively, in accordance with some embodiments described herein.

FIG. 24A depicts Pink-fit LFP power (5-100 Hz) averaged over 10 mins intervals through the course of first aCSF infusion (beginning at 0 min), and muscimol infusion (beginning at 30 mins) from signals recorded in FIGS. 21A and 21B in accordance with some embodiments described herein.

FIG. 24B depicts LFP power for different categorical spectral ranges (not including 53-65 Hz where line noise interferes with physiological signal) based on above plots as averaged over the 10 mins intervals as a function of time in accordance with some embodiments described herein.

FIGS. 25A-25C depicts normalized intensity vs. position curves at 5, 10, 15 and 20 mins for Cu-64 infusions (3 μCi/μl, 1.67 μl infusion at 10 μl/hr) delivered into an agarose phantom (FIG. 25A) using a syringe pump, in rat brain through implanted S-MiNDS using a syringe pump (FIG. 25B), and an iPrecio pump (FIG. 25C) in accordance with some embodiments described herein.

FIG. 26 depicts an SEM image of the tip of W-tetrode comprising four individual W electrodes (T-1, T-2, T-3, T-4) in accordance with some embodiments described herein.

FIG. 27A depicts the entire absorbance spectrum eluted over time through a 25 cm (L)×4.6 mm (ID) Spherisorb Column in accordance with some embodiments described herein.

FIG. 27B depicts relevant peak for muscimol concentration in accordance with some embodiments described herein.

FIG. 27C depicts muscimol stability results over time up to 54 days, at stock concentration of 0.2 mg/ml up to 6 serial dilutions (0.1, 0.05, 0.025, 0.0125, 0.00625, 0.003125 mg/ml) in accordance with some embodiments described herein.

### DETAILED DESCRIPTION

Systems, devices, and methods are disclosed herein for a biocompatible, remotely controllable, minimally invasive neural drug delivery system permitting dynamic adjustment of therapy with pinpoint spatial accuracy. In particular, the devices overcome manufacturing/assembly challenges previously encountered in creating devices that have both the needed mechanical strength and the micron scale, high aspect ratio dimensions needed to reach deep brain delivery sites in a minimally invasive manner.

In some embodiments, the system includes an electrode for neural activity recording for potential feedback control at a single-cell and population level, as well as two or more fluidic microtubes connected to two or more pumps configured to deliver liquids (containing a drug) at the nanoliter scale. In some embodiments, the system includes a tungsten (W) electrode for neural activity recording for potential feedback control at a single-cell and population level, as well as at least two fluidic borosilicate (BS) channels (microtubes) connected to wireless pumps for delivering nanoliters of drugs on demand. The microfabricated systems, devices, and methods provide therapeutic potential by allowing the monitoring of neural circuits at a single cell level while delivering nanodoses of therapeutic drugs to the brain.

These systems described herein may be referred as a Minimally-invasive Neural Drug delivery System, having the acronym “MiNDS”. It may be referred to as the “L-MiNDS” (for long) or as the “S-MiNDS” (for short). In addition, these MiNDs systems and their uses may also be referred to herein as “the system,” the “drug delivery system,” the “device,” the “drug delivery device,” the “drug delivery method,” and/or simply “the method.”

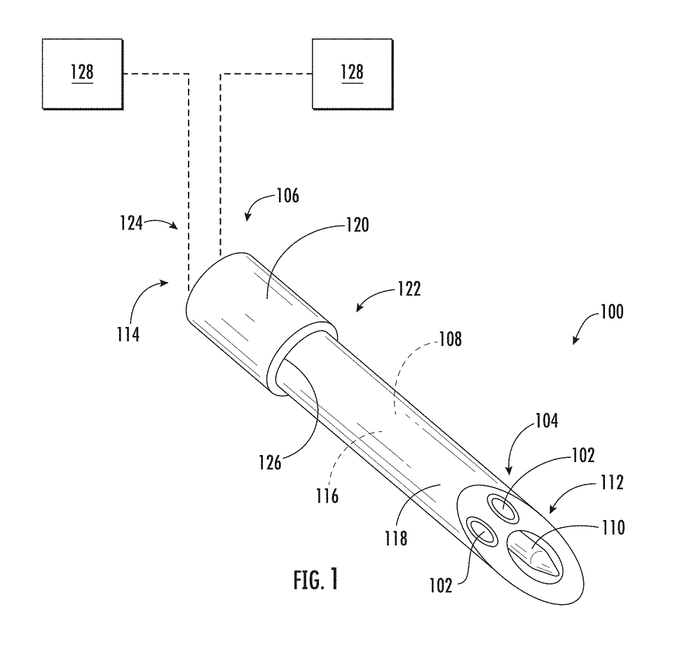

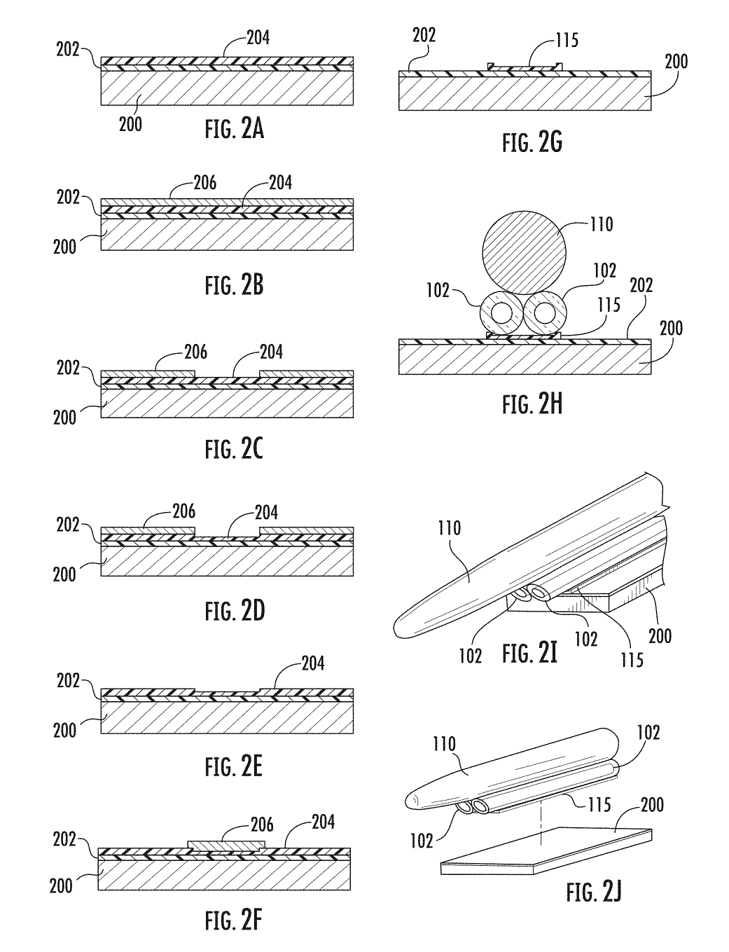

In some embodiments, as depicted in FIGS. 1 and 2, the drug delivery system 100 includes two or more discrete, annular microtubes 102. Each of the microtubes 102 comprises a distal end 104, a proximal end 106, and an elongate channel body 108 extending therebetween. In other instances, the drug delivery system 100 includes only a single microtube 102. The drug delivery system 100 may include any suitable number of microtubes 102. The drug delivery system 100 also includes an electrode 110 comprising a distal end 112, a proximal end 114, and an elongate body 116 extending therebetween, along with an elongated carrying template 115 supporting the microtubes 102 and the electrode 110 in an aligned stack. An annular needle 120 having a distal end 122 and a proximal end 124 is at least partially disposed about the carrying template. For example, the annular needle 120 includes an annulus 126 for housing the carrying template, the microtubes 102, and the electrode 110. In some instances, the drug delivery system 100 includes an aligner tip 118 for securing the distal ends of the electrode and the microtubes at a fixed position about the distal end of the annular needle 120. In some instances, the drug delivery system 100 includes at least one pump 128 fluidically connected to the proximal end(s) 106 of at least one of the microtubes 102. The pump 128 is configured to deliver a fluid drug on demand through the elongate channel body 108 and out of the distal end 104 of the one or more of the microtubes 102. In some instances, each of the microtubes 102 is in fluid communication with a respective pump of the at least one pump 128. That is, the number of microtubes 102 and the number of pumps may correspond. The drug delivery system 100 may be configured for delivery of the fluid drug to a neural tissue site in vivo.

In some preferred embodiments, the annular needle, the electrode, and the two or more microtubes have a length from about 1 cm to about 10 cm. The assembly of these components also preferably is very narrow, providing a high aspect, minimally invasive structure that can reach deep neural tissues site, such as in the brain. For example, in some embodiments, the annular needle has an aspect ratio (length:diameter) of at least 500. In one embodiment, the annular needle has an outer diameter of about 200 microns. In some other embodiments, the annular needle may have an outer diameter from about 150 microns to about 250 microns.

The microtubes serve as fluid conduits, i.e., infusion channels, and are sometimes referred to herein simply as “channels.” In preferred embodiments, the microtube is an annular structure with an annulus size small enough to minimize/eliminate diffusion of the drug fluid when the system is in the off state, thereby enabling pinpoint, sub-mm³ volume dosing. For example, in one embodiment, the microtube has an outer diameter of about 30 microns and an inner diameter of about 20 microns. The microtube may be formed of any suitable material, such as a biocompatible material that is also compatible with the drug fluid. In some preferred embodiments, the microtube is formed of a borosilicate glass. In some other embodiments, the microtube may be formed of silicon nitride, aluminum nitride, silicon dioxide, quartz, polyimide, polyurethanes, silicon rubber, polyethers, polyesters, co-polymers of polyether urethanes, polyester urethanes, polysulfones, polybutadiene-styrene, elastomers, copolymers of polylactide-co-glycolide, ethylene-acrylate rubber, polyester urethane, polybutadiene, chloro isobutylene isoprene, polychloroprene, chloro sulphonated polyethylene, epichlorohydrin, ethylene propylene, polyether urethane, perfluorocarbon rubber.

Any suitable electrode may be used. In some embodiments, the electrode includes one or more biocompatible metal wires with an electrically insulating oxide coating between its/their proximal and distal ends. In some instances, the electrode is a tungsten electrode. In some embodiments, the electrode is a tetrode.

In certain embodiments, the annular needle provides high bending stiffness, enabling the distal ends of the microtubes and electrode to be inserted into tissue at precise locations while housing and protecting the relatively fragile microtubes within the annulus of the needle. The annular needle is formed of and/or coated with a biocompatible material. In a preferred embodiment, the annular needle is formed of a stainless steel alloy. Other metals and other materials of construction also may be suitable. In some embodiments, the annulus of the needle has an inner diameter sized to accommodate the elongate carrying template supporting an aligned stack that includes the electrode and one, two, three, or four microtubes. In some embodiments, the annular needle has an outer diameter of about 200 microns.

In certain embodiments, the elongate carrying template is a microfabricated structure configured to support and secure the microtubes and electrode so that they can be assembled together within the annular needle. The elongate carrying template is useful in preventing or at least reducing fracture of high aspect ratio microtubes (formed of relatively brittle materials, such as borosiliate) during the assembly process. In one embodiment, the carrying template comprises a polyimide structure. In some other embodiments, the carrying template may be made of one or more other materials or composites. In preferred embodiments, the carrying template is sufficiently rigid and shaped to evenly support the microtubes stacked on top of the carrying template. In some embodiments, the carrying template includes a substantially flat elongated base and a pair of sidewalls on opposed sides of the base. The side walls and base define an elongate groove in which one more microtubes and one or more electrodes can be stabilized, for example, in a stack. In one embodiment, the sidewalls have a height effective to keep the microtubes from rolling off of the base, and the width of the groove is effective to hold two microtubes side-by-side.

In embodiments, the drug delivery system further includes an aligner tip securing the distal ends of the electrode and the two or more microtubes at a fixed position about the distal end of the annular needle. In a preferred embodiment, the aligner tip is formed of borosilicate, although other biocompatible materials of construction are envisioned.

A method is provided for local delivery of a fluid drug into a patient in need thereof. In some embodiments, the methods includes: (a) inserting the distal ends of the annular needle, the electrode, and the two or more microtubes of the described drug delivery system into a selected target tissue site in the patient; and then (b) delivering one or more doses of the fluid drug to the selected tissue site via at least one of the microtubes of the drug delivery system. As used herein, the term “patient” generally refers to a human or other mammal. The selected target tissue site may be any suitable neural tissue. In some embodiments, the target tissue site may be a neural network site, for example, in a patient's brain.

A method is provided for neural circuit modulation in a patient in need thereof. In some embodiments, the method includes (a) inserting the distal ends of the annular needle, the electrode, and the two or more microtubes of the drug delivery system described herein into a neural network site in the patient; and (b) delivering one or more doses of the fluid drug to the neural network site via at least one of the microtubes of the drug delivery system. The neural network site may be in the patient's brain. In one embodiment, the neural network site is in a deep brain structure.

In these methods, each of the one or more doses of the fluid drug may be a bolus of any suitable small volume. In some embodiments, the dose is from about 17 nL to about 2 μL. For example, the dose volume may range from 17 nL to 1.5 μL, from 17 nL to 1 μL, from 17 nL to 100 nL, from 17 nL to 200 nL, from 17 nL to 500 nL, from 20 nL to 750 nL, from 50 nL to 500 nL, or from 50 nL to 1.5 μL.

In these methods, the fluid drug may include a neuromodulating agent. In some embodiments, the neuromodulating agent comprises muscimol or another GABA agonist. Other neuromodulating agents known in the art also may be used.

The present drug delivery systems improve over the injectrodes described in U.S. Patent Application Publication 2016/0166803, which is incorporated by reference herein in its entirety. For example, in some embodiments, the present systems provide more easily manufactured and assembled fluidic components; improved mechanical robustness for penetrating in deep brain structures without mechanical failure, whilst maintaining minimal invasiveness; and increased mechanical robustness of the fluidic connections on the proximal end of the system, coupling more easily to conventional drug delivery pumps without leakage.

Advantageously, the system is customizable in multiple ways depending on the desired application. This minimally invasive, microfabricated device with high bending stiffness, high aspect ratio and adjustable number of channels (i.e., multi-functionality) in the annular needle, allows the targeting of deep brain structures and the reliable modulation of both local neural activity with cell-type specificity and behavior dependent upon this activity. The number of fluidic channels (microtubes) may be increased to deliver a variety of drugs as well as to insert optical fibers, all are incorporated, to perform optogenetics. The small size of the fluidic channels (e.g., OD=20 μm) proves to minimize diffusion when the system is in the off state with negligible system compliance. Additionally, the system includes fine, localized and bidirectional infusion capabilities. The number, material, or type of the electrode inside the system can also be changed. Given the wall thickness of stainless steel can be tuned via chemical etching, the bending stiffness of the system can be readily engineered depending on the target tissue. Moreover, the system can be used in other applications besides neuroengineering. By adjusting its length, stiffness and available channels, the system can selectively provide drugs or light or electricity to specific organs of the body with pinpoint spatiotemporal resolution.

In this manner, the disclosure is directed to the neuroengineering of a minimally invasive neural drug delivery system. The system may include a diameter of about 200 μm and an aspect ratio of about 500. In some instances, the system may be integrated with a tungsten (W) electrode to record neural activity for potential feedback control at a single-cell and population level. The system also may include at least two fluidic channels (microtubes) connected to modified wireless pumps, such as iPrecio® pumps, for delivering nanoliters of drugs on demand. Any suitable pump may be used herein. The system may have functioning capability over at least two months or longer. The system has been tested respectively in small (i.e., rodent) and large (i.e., non-human primate (NHP)) animal models to demonstrate chronic behavioral and acute electrophysiological effects.

The present systems provide the capacity selectively to deliver drugs on demand to brain structures that are of the order of 1 mm³, which may greatly improve therapeutic outcome and minimize unwanted side effects over currently available methods. In addition to treating neurological disorders, these microfabricated devices and systems described herein may be used to deliver chemicals, light, and electricity to other organs and to tumors with pinpoint spatiotemporal resolution.

The systems and methods can be further understood with reference to following non-limiting examples.

### EXAMPLE 1

### Making a Neural Drug Delivery Device

Fabricating the Polymer Template

As depicted in the fabrication sequence shown in FIGS. 2A-2J, a silicon (Si) wafer 200 was coated with a 50 nm thick layer of poly(methyl methacrylate) 202 (PMMA 495 A2) at 3,000 rpm (Headway Research, PWM32) for 30 s, and baked on a hotplate at 180° C. for 2 mins. A poly(pyromellitic dianhydride-co-4,40-oxydianiline) amic acid solution was then spun at 4,000 rpm for 30 s, and pre-cured on a hotplate at 150° C. for 1 min to form a 1.3 μm-thick polyimide (PI) layer 204. This step was repeated for seven times to reach a ˜9.2 μm thick layer of PI. Next, the sample was cured in a vacuum oven at 250° C. for 1 hr. The walls of the PI template (depth of 2.8 μm) were formed by reactive ion etching (March RIE, Nordson) through a pattern of PR 206 (AZ 4620—Clariant) until the layer of PMMA was reached on the Si wafer (FIGS. 2A-2J). The length of the PI template was set to 7 cm. This component was the elongate carrying template.

Customizing the Stainless Steel (SS) Needle for the MiNDS

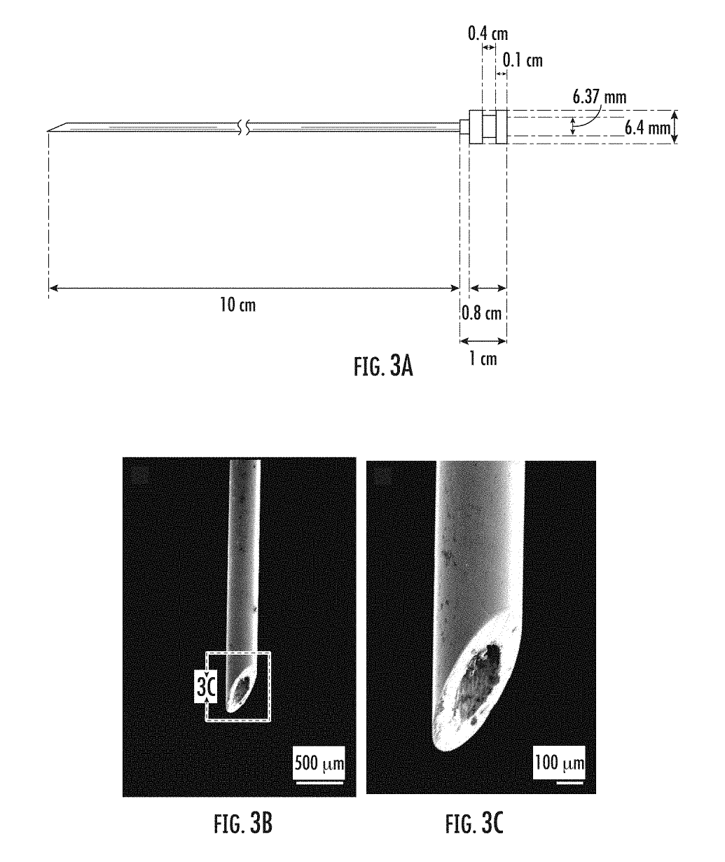

The outer diameter of a SS needle (30G, Hamilton Company) was etched to 200 μm via a chemical solution of 10 wt % ferric chloride (FeCl₃), 10 wt % hydrochloric acid (HCl) and 5 wt % nitric acid (HNO₃) at 50° C. with an etching rate of 2 μm/min. To protect the inner wall of the SS needle from the chemical solution, a PI tubing was placed tightly inside and then removed after the etching process (FIGS. 2A-3C). This component was the annular needle for housing the microtubes, carrying template, and electrode.

Polishing the Tip of the Borosilicate (BS) Channel to a 30° Angle

A BS channel with an inner diameter of 20 μm and an outer diameter of 80 μm (VitroCom Inc.) was firstly etched down to 30 μm via a chemical solution of hydrofluoric acid (HF 48%, Sigma Aldrich) in deionized (DI) water (volume ratio of 1:2) with an etching rate of 6 μm/min. The ends of the BS channel were protected via three layers of polyimide tape (CAPLINGQ Corporation, 20 μm thick). Afterwards, the BS was placed into the polisher holder, angled to 30° and positioned until the tip touched the polishing film (8 inch diameter aluminum oxide, Al₂O₃, polishing film, ULTRATEC Manufacturing Inc.). The lap speed was set to 250 rpm. After the tip of the BS channel was polished for ˜2 hrs, it was immersed into water in an ultrasonic cleaner (KENDAL, Model CD-3800A) for ˜3 mins to clean the remaining residues. This BS channel was the microtube component.

Electrical Insulation of the Tungsten (W) Electrodes

A dielectric stack of silicon dioxide (SiO₂) (50 nm)/aluminum oxide (Al₂O₃) (10 nm)/SiO₂ (50 nm) was deposited on the W electrode (FHC Inc.) via plasma enhanced chemical vapor deposition (PECVD, Plasmatherm System VII) and atomic layer deposition (ALD, Cambridge NanoTech Inc.) respectively to provide the electrical insulation. The exposed W electrode tip (˜9,700 μm²) was defined by dipping the wire into polyvinyl alcohol (PVA) solution with a depth of ˜25 μm. After the dielectric stack deposition, the protective layer of PVA on the W tip was dissolved in a water bath. This was the electrode component.

Aligning the Channels on the PI Template and Assembling the MiNDS

A 2 cm thick stamp of polydimethylsiloxane (PDMS, Sylgard 184), with a length and a width of 8 cm and 2 cm respectively and a mixing ratio of 8.5:1.5 base to crosslinker, was used to pick up the borosilicate (BS) channels and the W electrode gently, and align it with the PI template, as depicted in FIGS. 2H and 2I, under optical microscopy with the help of a mask aligner (Karl Suss Model MA4).

Two BS channels were then aligned side by side on the PI template, and the W electrode was placed on the center of the glass tubes. A ˜3 μm thick layer of UV light curable silicone adhesive (UV epoxy, LOCTITE 5055™, Henkel Corp) coated the PI template and covered both the BS channels and the W electrode inside a desiccator. Once the epoxy was cured, the PI template with the BS channels and the W was immersed in a hot (85° C.) acetone bath to allow the sacrificial layer of PMMA to dissolve away, as depicted in FIG. 2J. The epoxy coated PI template was then physically free and could be retrieved from the acetone bath. Afterwards, the PI was aligned with the polished end of the SS needle hole and aligned along the needle hole by using a vacuum tweezer (Ted Pella, Inc., Vacuum Pickup System, 115 V), which holds the template gently from the other end with a vacuum of 20″ of mercury.

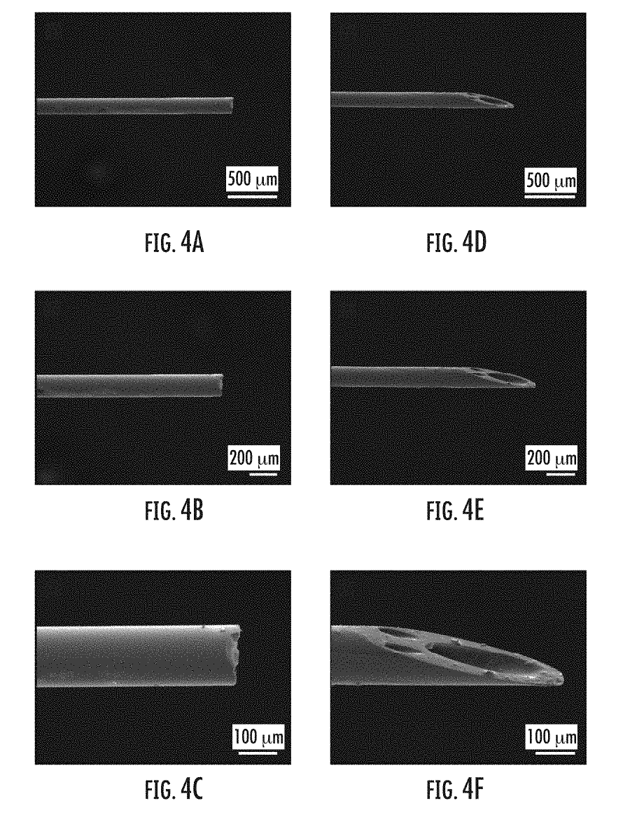

A customized BS tip aligner (VitroCom Inc.) was obtained containing two 35 μm and one 90 μm diameter channels to serve as the alignment of the BS channels and the W electrode, respectively. The tip of the BS tip aligner was also polished to an angle of 30°, as depicted in FIGS. 4A-4F. The length of the BS aligner tip can be engineered, and the other end was polished with an angle of 0°. Later, the blunt end of the BS aligner tip was aligned with the expanded W electrode and attached to the pre-cured epoxy layer in the tip of the SS needle.

The fluidic connections to connect the BS channels to the wireless pumps were created via polyether ether ketone (PEEK) tubes (Tub Radel R, IDEX Health & Science LLC, 0.0625″ outer diameter×0.10″ inner diameter) and followed by UV epoxy sealing.

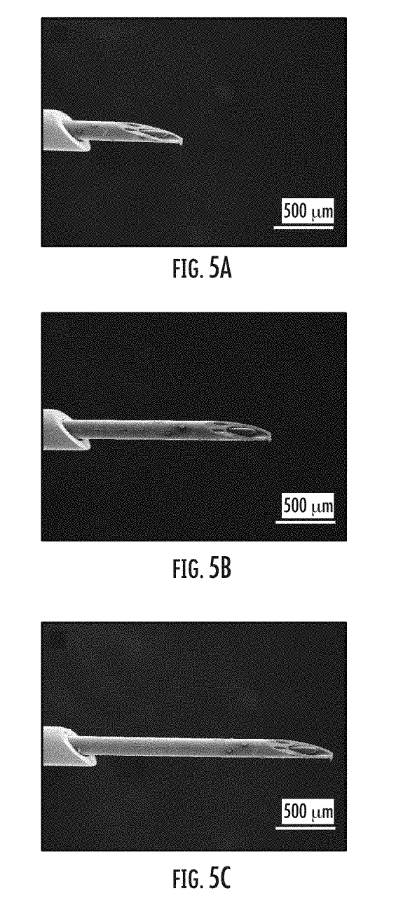

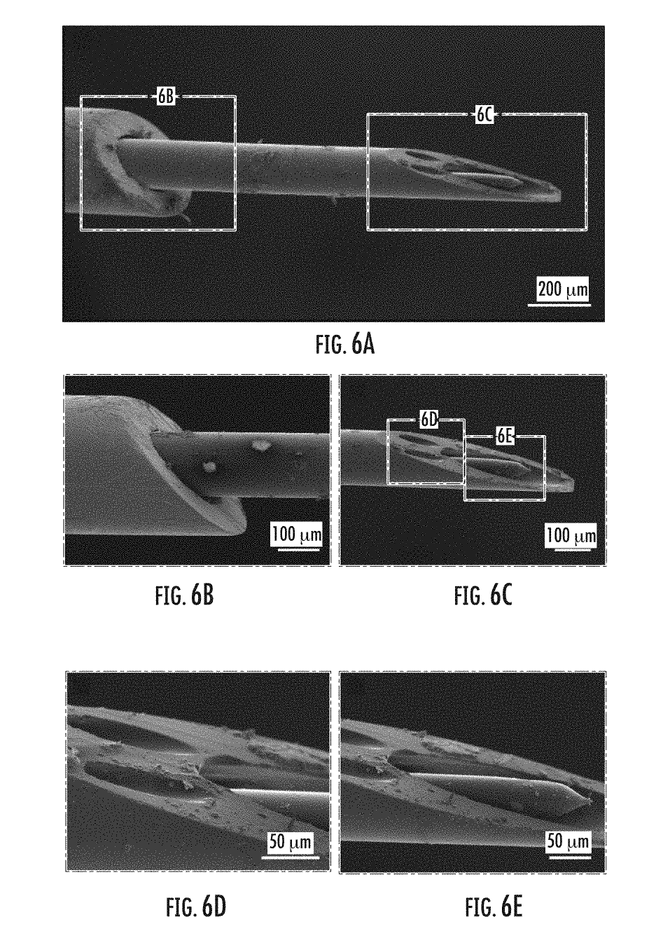

The fluidic connection was made by aligning the PEEK tubings with the flexible BS channels in the metal cup of the SS needle under microscope and filling all the gaps with UV epoxy with a connection yield of ˜%100. The electrical connection to the W electrode was made via a metal pin (Conn Recept Pin, Mill-Max Manufacturing Corp, 0.300″ length, 0.015″˜0.022″ accepting pin diameter, 0.037″ mounting hole diameter, 0.031″ pin hole diameter, 0.041″ flange diameter, 0.018″ tail diameter, 0.150″ socket depth). The UV epoxy was then used to fill the gap between the PI template and the SS hole via vacuum tube, sucking from one end and filling with epoxy on the other end of the SS. As the MiNDS is scalable, its length can be modified according to the desired subject application, as depicted in FIGS. 5A-6E.

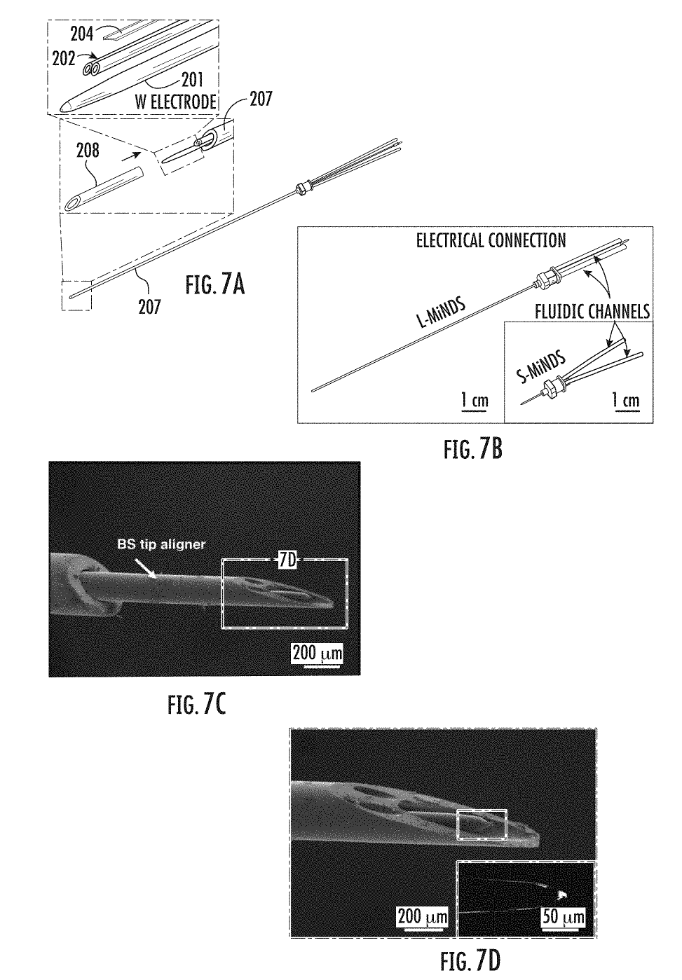

FIG. 7A shows a schematic diagram of the system with magnified and exploded views of the tip. As depicted, the system includes a tungsten (W) electrode 201 (having a diameter of about 75 μm), at least two BS channels 202 (each has an outer diameter (OD) of about 30 μm and an inner diameter (ID) of about 20 μm), and a PI template 204 (having a thickness of about 9.2 μm) that are all aligned with a vacuum tweezer inside an etched stainless steel Hamilton needle 207 (OD=200 μm, ID=150 μm). Stainless steel was chosen as the backbone of the system because it is mechanically robust, can be easily etched, and is compatible with chronic use in brain implants. However, other suitable materials may be used. Furthermore, the system is scalable, with length modifiable according to the desired application, as depicted in FIG. 7B, where the length of S-MiNDS and L-MiNDS are about 1 cm and 10 cm, respectively. The system may be any other suitable length, however.

Scanning electron microscopy (SEM) images of the tip of the system are depicted in FIGS. 7C, 7D, which demonstrates the BS aligner tip 208 with a tip angle of about 30°, which has an outer diameter of about 150 μm, composed respectively of two 35 μm and one 90 μm diameter openings for individual BS channels and W electrode. The BS aligner tip serves as a protective confined layer for the tip of the system and is aligned with individual BS channels and W electrode, as depicted in FIG. 7A, capitalizing on BS being a biocompatible material in the brain and being readily chemically etched. FIGS. 7C and 7D illustrates the tip of a W electrode with a dielectric stack of silicon dioxide (SiO₂) (50 nm)/aluminum oxide (Al₂O₃) (10 nm)/SiO₂ (50 nm) as an electrical insulation layer for the regions that are ˜25 μm away from the electrode tip.

### EXAMPLE 2

### Resistivity Measurements

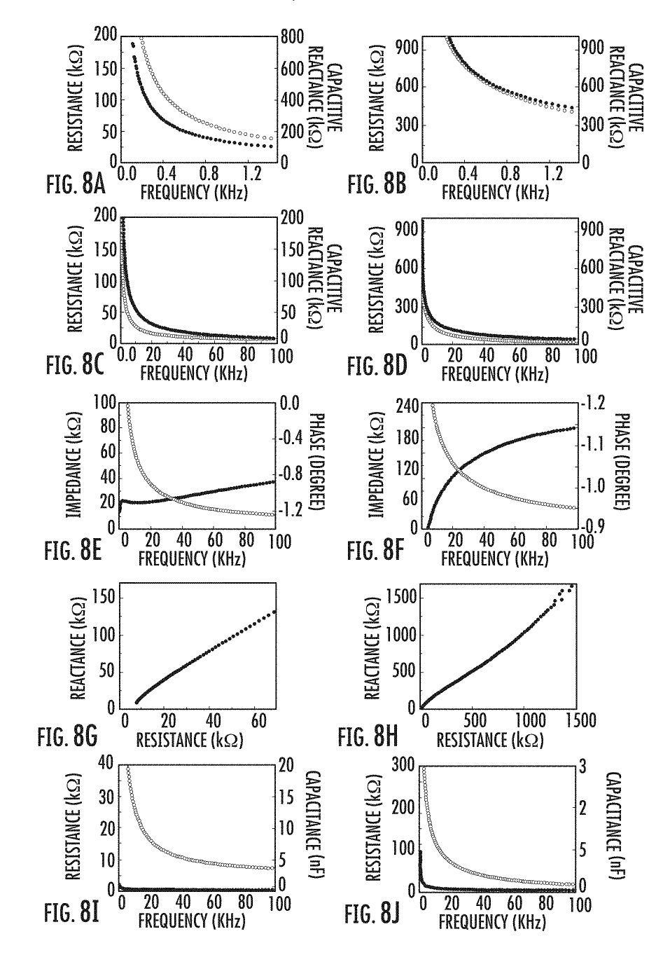

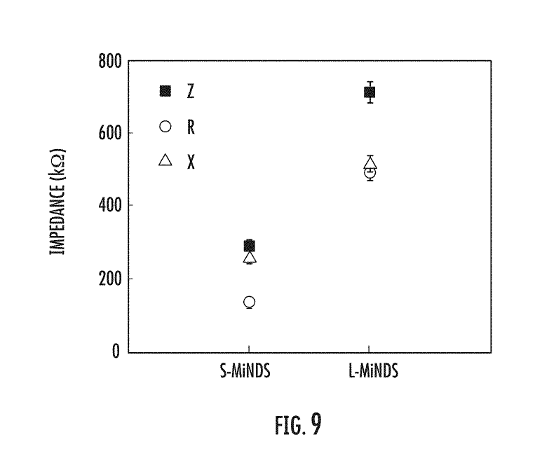

An impedance measurement system (Keysight E4980A) was used to measure the resistance and reactance of the S- and L-MiNDSs at 5 mV with a frequency sweep of 201 data points from 100 Hz to 100 kHz. The measurements were performed by submerging the tip of the MiNDS into a saline bath (0.9% sodium chloride, Baxter) and connecting W electrode to one of the analyzer terminals. The second analyzer terminal was submerged in the same saline bath, ensuring no physical contact with the MiNDS. The impedance testing for each of the MiNDSs was repeated four times to estimate the error of the measurements, as depicted in FIGS. 8A-8J. FIGS. 8A and 8B depict resistance-capacitive reactance vs. frequency graphs at high frequency for S-MiNDS and L-MiNDS, respectively. FIGS. 8C and 8D depict resistance-capacitive reactance vs. frequency graphs at low frequency for S-MiNDS and L-MiNDS, respectively. FIGS. 8E and 8F depict impedance-phase (degree) vs. frequency graphs for S-MiNDS and L-MiNDS, respectively. FIGS. 8G and 8H depict reactance vs. resistance graphs. FIGS. 8I and 8J depict resistance-capacitance vs. frequency graphs for S-MiNDS and L-MiNDS, respectively. The calculated error bars represent the standard errors for the S- and L-MiNDSs, as depicted in FIG. 9.

### EXAMPLE 3

### Pump In Vitro Infusion Characterization

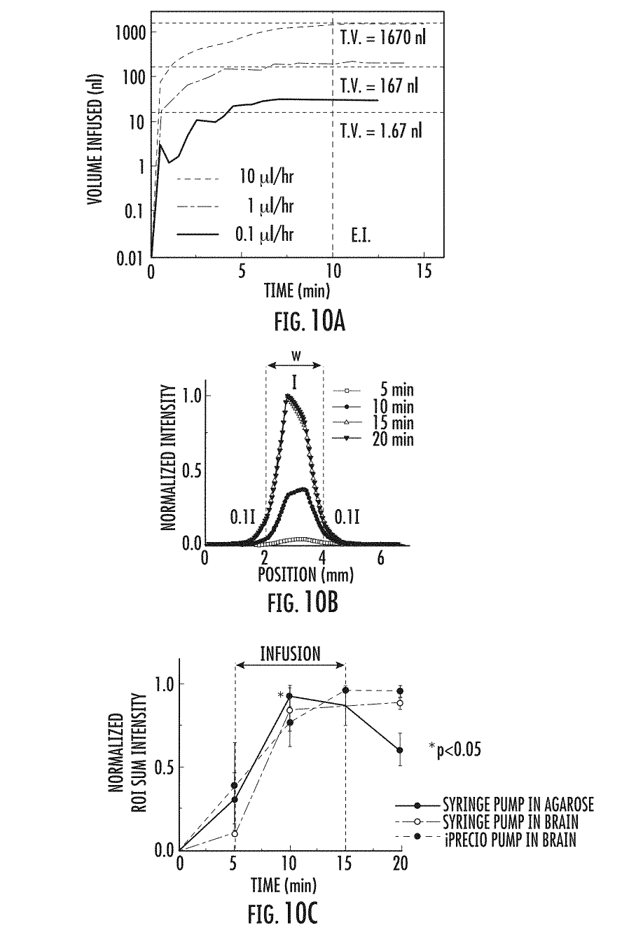

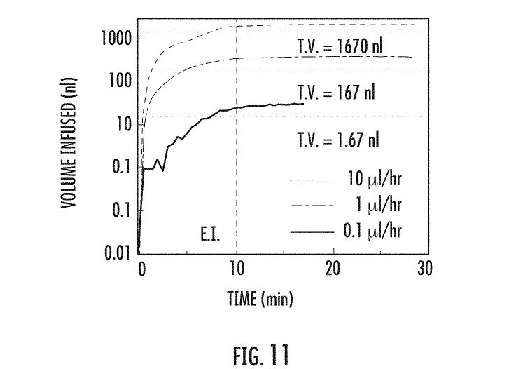

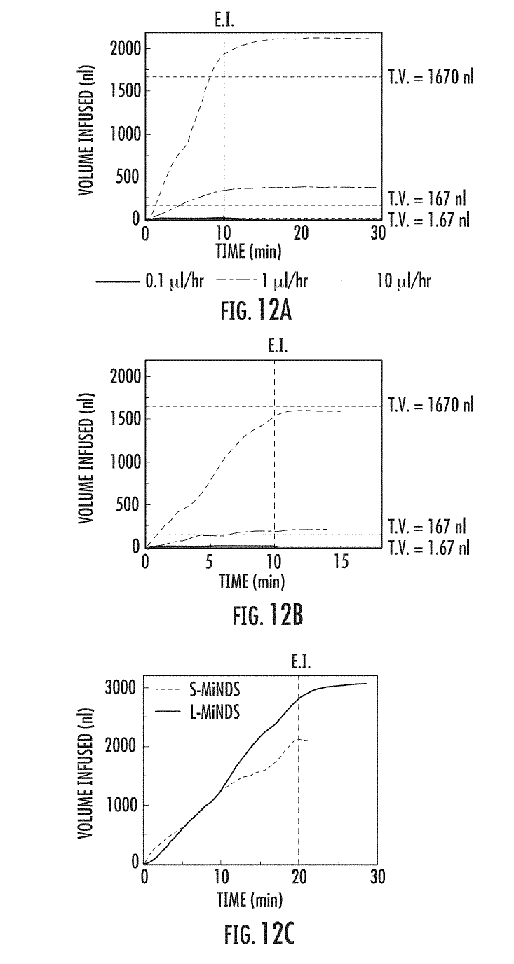

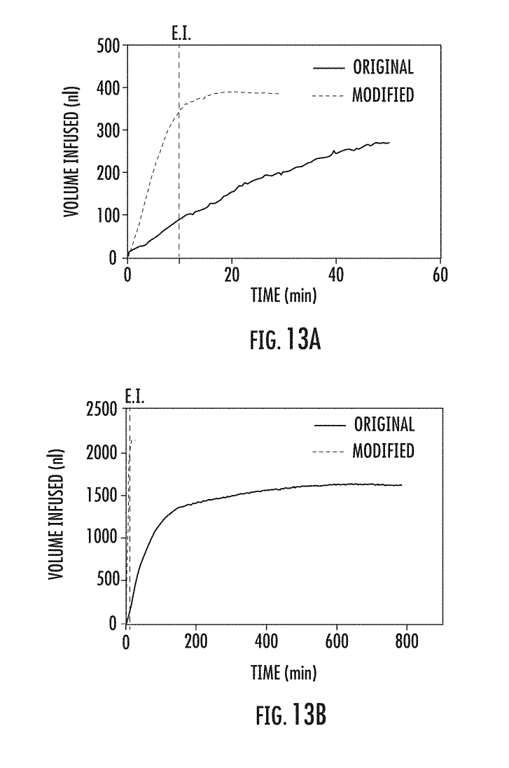

The electrode and microtube assembly was connected to two independently controlled, modified, SMP-300 iPrecio pumps and a precision microbalance was used to determine the in vitro behavior of the system. The infused media was DI water with density of 1 kg/m³. The average infusion profile of the S-MiNDS system for 10 mins infusion profiles at the flow rates of 10, 1, and 0.1 μl/hr can been seen in FIG. 10A. The system performed optimally with 3.3% accuracy at the rate of 10 μl/hr infusion, as depicted in FIGS. 11 and 12A-12C. No infusion past the programmed end of pumping was noted, indicating that the compliance of the system is negligible and that there is negligible passive leakage of fluid out of the BS channel. The extremely small size of the fluidic channels (ID=20 μm) minimized diffusion when the system was in the off state. Thus, dosing using the system can be acutely turned on and off, demonstrating the reliable and consistent functionality of the system to achieve repeated local targeting of deep brain regions. As depicted in FIGS. 13A and 13B, modification of the iPrecio pumps to minimize system compliance was used to ensure reliable on/off dosing.

In order to achieve fine control over volume delivery, the original high compliance thermoplastic infusion tubing (Elastic Modulus 1 MPa-200 MPa) of the pump was replaced with high pressure, low compliance PEEK tubing (Elastic Modulus 3.6 GPa) and stainless steel adapters. For each of the two iPrecio SMP-300 pumps (Primetech Corp., Japan) used, the original external tubing was cut, leaving only the first 2.5 mm of outlet tubing. A 24G stainless steel connector was inserted into the pump fluid outlet, and a 31G connector was placed within the larger connector. The two steel connectors were glued together using UV epoxy, creating a water-tight secure junction. The protruding end of the 31G connector was inserted into the PEEK tubing of the MiNDS, and the junction was again glued with UV epoxy. The MiNDS was placed into a custom-made polytetrafluoroethylene (PTFE) holder (manufactured with CNC Micro Machining Center-S, Cameron Micro Drill Presses, Sonora, Calif.) and attached to a syringe pump to be used as a vertical frame (Harvard Apparatus PHD 2000). The assembly was combined with a computer controllable Mettler Toledo microbalance for pump characterization experiments.

A plastic weighing dish was made by cutting the needle cap of a 28G blunt needle using a stainless steel blade. The dish was half-filled with DI water (˜30 ml) and placed on the weighing plate of the microbalance. The glass cap of the microbalance was removed and parafilm was stretched over the top. A circular hole was cut out in the center of the parafilm using scissors. The MiNDS was lowered through the hole, and the pump set to infuse fluid until a drop of fluid appears at the top of the MiNDS. At this point, the device was lowered further down until the tip of the MiNDS was submerged in the water of the weighing dish. To minimize water evaporation, a 20 ml mineral oil layer (paraffin oil and liquid petrolatum, Mallinckrodt Chemicals, Dublin, Ireland) was placed on top of the water of the weighing dish. The system was allowed to stabilize before any infusions were tested. The pump was programmed wirelessly.

The microbalance was set to read output twice per second and send the data to a computer via a RS232 serial connector. Commercially-available Advanced Serial Data Logger software (AGG Software) was used to acquire the data and export it to Microsoft Excel for further analysis. For every infusion, data recording begins and ends at least 10 mins before and after infusion onset and end, respectively. This process was repeated for both long (L) and short (S) MiNDS, and each infusion protocol was run for 4 times. The 4 infusion protocols were run for each device: (1) 10 μl/hr for 10 mins, (2) 1 μl/hr for 10 mins, (3) 0.1 μl/hr for 10 mins, (4) 6 ml/hr for 20 mins. Infusion profiles are shown in FIGS. 10A, 11, and 12A-12C.

### EXAMPLE 4

### Chronic In Vivo Biocompatibility Assessment

Four rats underwent the MiNDS implantation. At 56 days post-implantation, the animals were euthanized using carbon dioxide asphyxiation. Each animal consequently underwent cardiac perfusion of 60 ml 1× phosphate buffered saline (PBS) solution (Corning Inc., Corning, N.Y., USA), followed by 60 ml 4% paraformaldehyde (PFA) solution (Alfa Aesar, Ward Hill, Mass.). The head was then removed and immersed in 4% PFA for 48 hrs. The implanted devices were extracted, and the brain removed and placed in 4% PFA overnight, and subsequently in sinking solutions of increasing sucrose (Amresco Inc., Solon, Ohio, USA) concentration (10%, 20% and 30% w/v) overnight or until the brain sinked. All animal protocols were approved by the MIT Committee for Animal Care (0714-072-17).

Histology Protocol for Chronic In Vivo Biocompatibility

The brain was embedded in frozen tissue embedding medium (Sakura Finetek USA, Torrance, Calif.), and frozen in a liquid nitrogen bath. 20 μm transverse slices were cut using a Leica CM1900 cryostat (Leica Biosystems Inc., Buffalo Grove, USA), starting at the top of the brain, and descending 80 μm, past the tips of the previously implanted devices. Slides were stored at −80° C.

Slides were removed from the −80° C. freezer, placed at room temperature for 20 mins, rehydrated by placing them in a 1× PBS solution for 10 mins. and then stained for astrocytes (glial fibrillary acidic protein (GFAP)), microglia (Iba1), neurons (NeuN), and nuclei (Hoechst/DAPI). Samples were then immersed in a blocking solution (5% Bovine Serum Albumin (BSA) (Rockland, Limerick, Pa.)) for 50 mins, followed by overnight incubation at 4° C. in a primary antibody incubation solution (1:100 mouse anti-GFAPx488 Alexafluor, 1:300 rabbit anti-NeuN, (EMD Millipore, Billerica, Mass., USA), 1:300 goat anti-Iba1 (Abcam, Cambridge, Mass., USA) in an incubation buffer (1% BSA, 1% normal Dk serum, 0.3% Triton X-100, 0.1% Sodium Azide).

Slides were rinsed 3 times in 1× PBS (0.1% Tween), and incubated in a secondary antibody solution (1:300 Dk×Gt×Cy3 & 1:300 Dk×Rb×Dy650 (Abcam) for 40 mins. Samples were rinsed three times in 1× PBS, and incubated with a Hoechst solution (0.1 μg/m1) for 5 mins, followed by mounting in a gold antifade reagent (Life Technologies, Carlsbad, Calif. USA).

Data Analysis for Histology

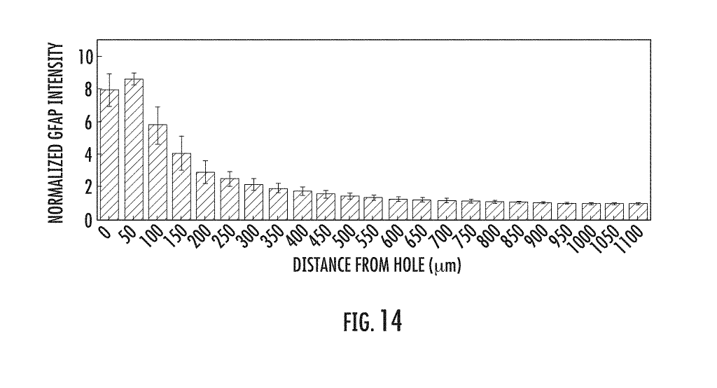

All images were taken using fluorescence microscopy (EVOS FL Auto, Life Technologies, Grand Island, N.Y.) and analyzed with custom MATLAB scripts. These scripts define the boundary of the hole created by the MiNDS increments from the hole boundary, up to 1100 μm away from the edge of the hole. For the purposes of data analysis, GFAP intensities were used as the primary indicator for the extent of glial scar formation around the implant. The intensities were then averaged into 50 μm bins, and normalized such that the intensity 900-1100 μm away was equal to 1. This was done for 4 pictures for each animal, which were averaged to create a GFAP intensity vs. distance profile. The profiles for each of the four rats were then combined and averaged, as depicted in FIG. 14.

In vivo testing of the system was performed in a series of experiments. In confocal fluorescent microscopy analyses, no significant tissue gliosis was found in response to the systems 8-weeks post-implantation in brain tissue as evaluated in four rats. Inflammatory response was limited to the immediate surrounding of the SS. This result confirms the minimal invasiveness and chronic viability of the system in vivo.

### EXAMPLE 5

### In Vivo Functionality Experiments

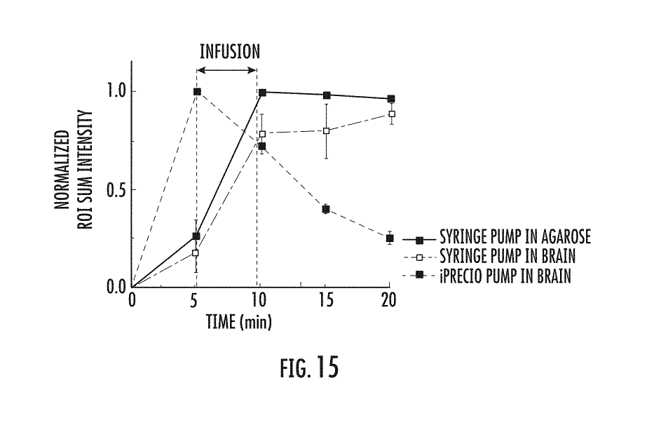

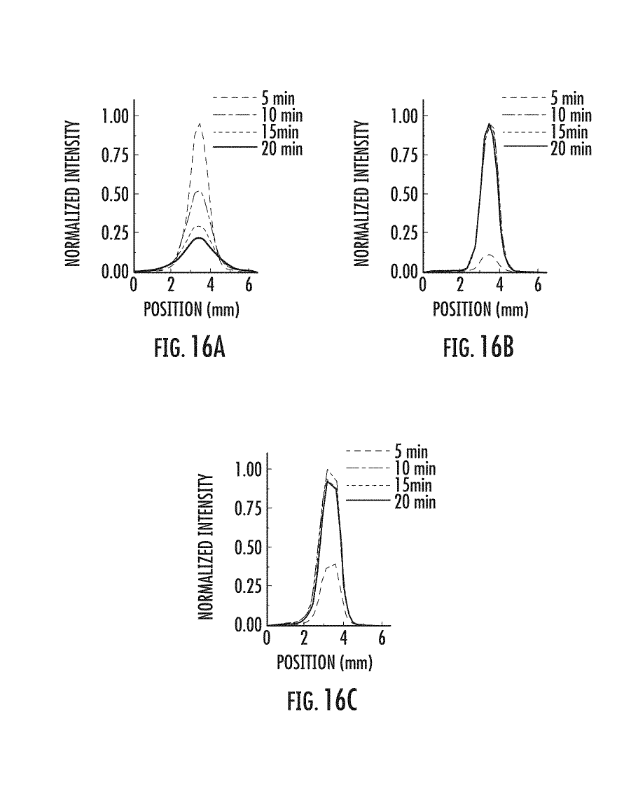

The functionality of the device was confirmed in the rat brain by positron emission tomography (PET) in vivo imaging, performing the use of 3D PET to visualize and characterize in vivo deep brain infusions, bringing in vivo testing to this field. A 0.6% (by wt.) agarose solution with an embedded S-MiNDS was used as a representative homogeneous brain phantom to perform the control trials. The in vivo case used an S-MiNDS chronically implanted in a rat and targeting the substantia nigra (SN), a brain region containing dopaminergic neurons. To validate the tunable infusion capability of the system, large and small infusions of Cu-64 were demonstrated: (1) 1.67 μl of Cu-64 (3 μCi/μl) over 10 mins and (2) 667 nl of Cu-64 (30 μCi/μl) over 4 mins. Accounting for the internal volume of the device, these infusions resulted in a 1.2 μl and 167 nl net volume delivered to the brain, respectively. A syringe pump was used to deliver infusions in the agarose control and in vivo, while iPrecio pumps were used only in vivo. The large infusion (1.67 μl) into the agarose control produced a bolus with a volume of 3.78 mm³+/−2.43 mm³. An identical large volume infusion delivered by a syringe pump in vivo produced a bolus volume of 4.64 mm³+/−1.43 mm³, while the in vivo iPrecio infusion resulted in a bolus with a volume of 4.36 mm³+/−0.45 mm³. FIG. 10B shows the line profile of this larger volume iPrecio infusion in vivo. The total intensity contained within the bolus for all cases was over 20 mins (FIG. 2F). Identical studies done with the smaller infusion (667 nl) formed a bolus volume of 2.35 mm³+/−1.14 mm³ in the agarose brain phantom and volume of 1.81 mm³ and 2.8 mm³+/−0.15 mm³ in vivo using the syringe and iPrecio pumps, respectively, as depicted in FIGS. 15-16C. In all tests, localized bolus delivery was observed with limited diffusion, as depicted in FIGS. 10B and 10C. These results demonstrate the capability of the system to control the delivery of small quantities of drug remotely to an animal without any tethering or physical connection. The time sequence of PET images acquired at various time points further show the capability of the system to maintain a localized bolus delivery. The collective infusion results show that the system significantly avoids the problems of backflow inevitably encountered in acute infusions, and can deliver nanoliter quantities of drugs in a tunable, repeatable manner.

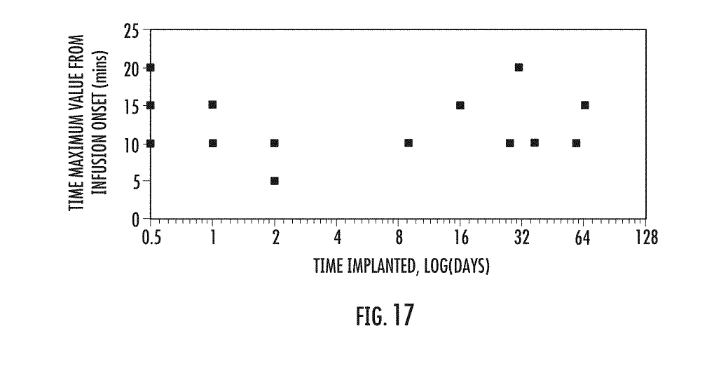

Infusions were confirmed using PET imaging in animals up to 65 days/weeks post-implantation. As depicted in FIG. 17, no delay in infusion was noted, suggesting that no significant resistance to infusion developed. This confirms that the system retains biocompatibility for chronic functionality in vivo.

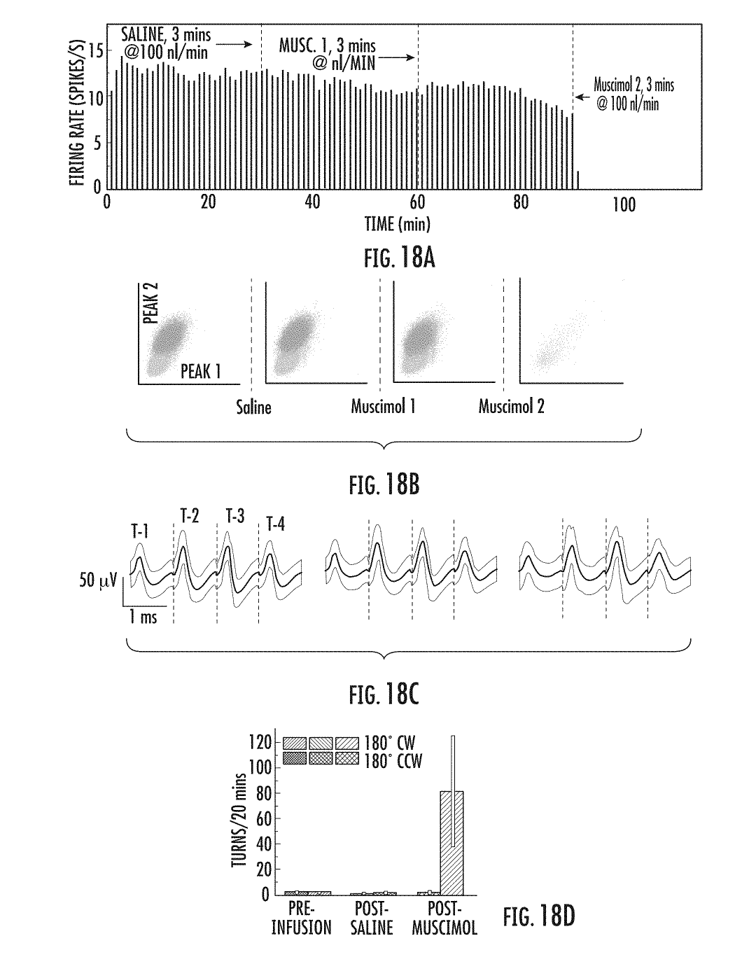

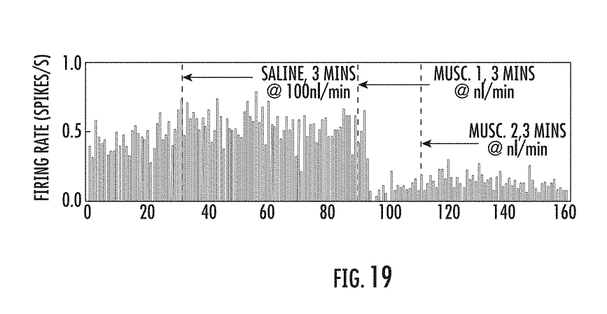

Acute electrophysiological recordings and micro-injections were performed in anesthetized rats to test the system with a tetrode W electrode. FIG. 18A shows the firing rate of a well isolated hippocampal (CA1) unit that was modulated by local infusion of muscimol, a GABA_{A} agonist and saline via two implanted iPrecio pumps. The channel impedance values of the tetrode electrode (T-1, T-2, T-3, T-4) were 430, 370, 440, and 370 kΩ. As expected, the injections of saline did not induce a significant change in the firing rate of the neuron. The firing rate of the unit was stable before the first injection of muscimol, after which a slow decrease of the firing rate occurred; then the second injection abolished the rate of detected spike activity. The mean of the action potentials recorded from this unit was stable during the experiment (i.e. before first saline infusion, before first and second muscimol infusions), confirming that the unit was present during the trial, as shown in FIGS. 18A-C. In FIG. 18A, the 1^{st}, 2^{nd} and 3^{rd} vertical line on left indicates the start of saline infusion (at 30 mins), the muscimol infusion (at 60 mins), and the second muscimol infusion (at 90 mins), respectively. FIG. 18B depicts representations of sorted (light) and unsorted (dark) action potentials based on peak values. Peak 1 and Peak 2 are the maximal value of waveforms measured by T-1, T-2, respectively. The projections of the peak values calculated from each recorded action potential are shown in FIG. 18C, reflecting the cluster-cutting used to isolate signals coming from different neurons. The stability of the recorded neurons during multiple injections did not affect the shape of their action potentials. The firing rate of hippocampal cells modulated by the local injections of muscimol, a GABA_{A} agonist was confirmed in a second experiment, as depicted FIG. 19, which evoked similar firing rate modulations. The 1^{st}, 2^{nd}, and 3^{rd} vertical line on left demarcates indicates the start of saline infusion (at 30 mins), the muscimol infusion (at 60 mins), and the second muscimol infusion (at 90 mins), respectively

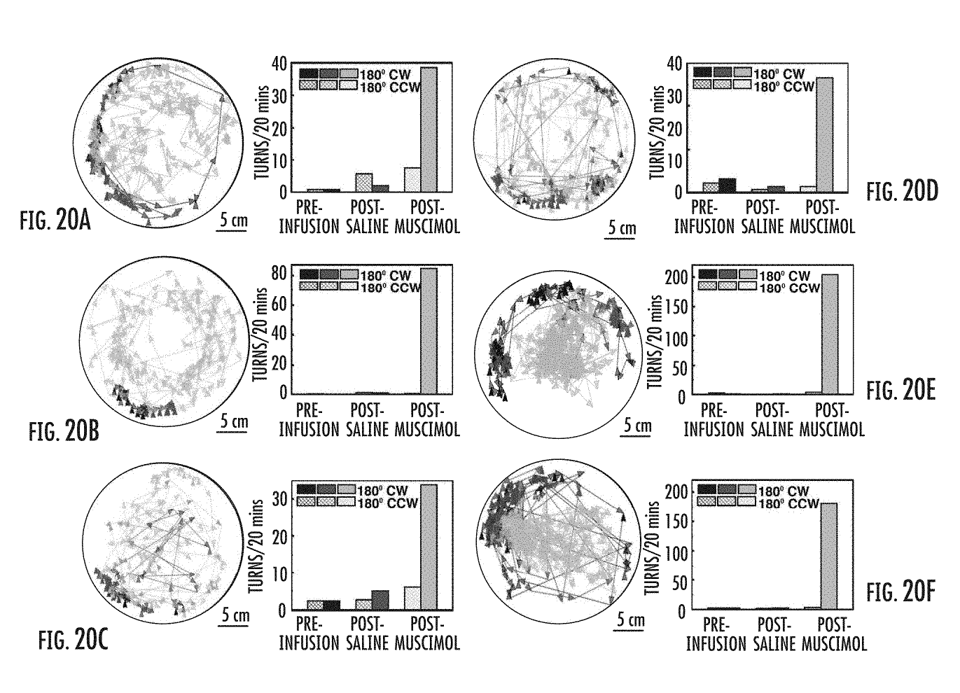

The capability of interfacing the system with deep brain structures to remotely control behavior was tested. In experiments in rats, the S-MiNDS was implanted in the SN and was connected to two implanted iPrecio pumps, containing either saline or muscimol. Unilateral delivery of muscimol to the SN is known to evoke preferential ipsilateral rotation, reflecting a hemiparkinsonian state. This parkinsonian behavior was reliably induced through remotely controlled infusion of 1.67 μl of muscimol (0.2 mg/ml) through the system, but not by comparable injection of saline, as depicted in FIG. 18D. The rat exhibited a 52-fold increase in the number of clockwise rotations while counter-clockwise rotations remained the same, as depicted in FIGS. 20A-20F. These studies were performed multiple times on each of multiple animals. This illustrates the ability of system to repeatedly and reproducibly deliver small volumes of drug to effect a reversible behavioral change.

Abnormal activity in neural circuits underlies many neurological disorders, which potentially could be treated by chemical or electrical stimulation of specific brain regions. Mood and anxiety related neuropsychiatric conditions can be modulated by neural projections from the neocortex to the basal ganglia. Human imaging and awake, behaving non-human primate experiments implicate that a brain region called the anterior cingulate cortex (ACC) is in the motivational/emotional regulation of behavior. The delivery of drugs or electrical current to the striatum has also proved to alter mood behavior in animal and human experiments. Here, the system represents a step towards providing new routes to deliver chemicals to specific regions of brain with pinpoint spatiotemporal resolution. By observing how the target region activity changes, the amount of the drug infusion can be further controlled as needed. Rodent studies presented here are the first instance to use a wirelessly controlled drug delivery system to elicit a reversible behavioral change in this model.

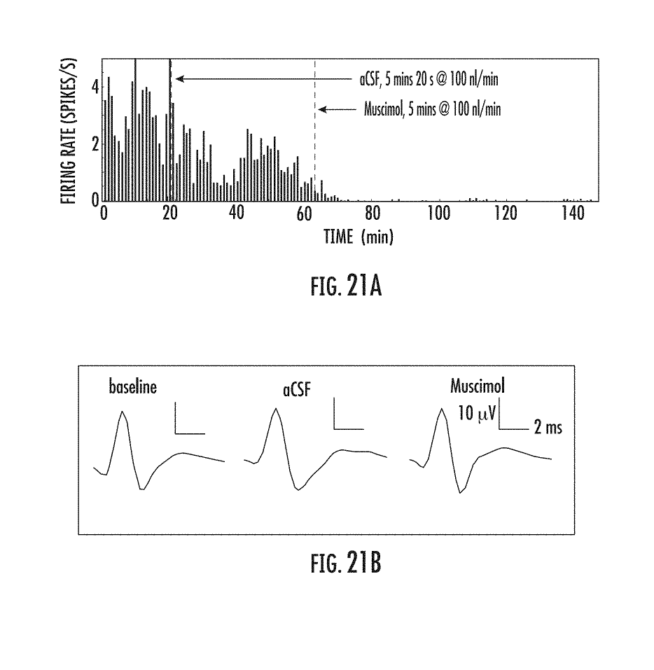

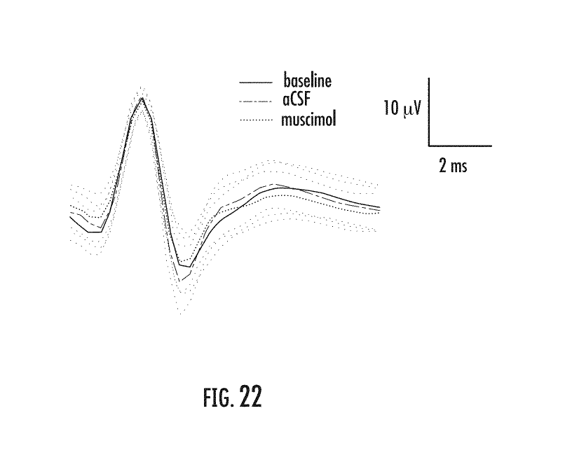

The functionality of the system (specifically, the L-MiNDS) was confirmed in a large, awake behaving animal model, the rhesus macaque (macacca mulatta) monkey. The L-MiNDS was used to modulate and monitor local neuronal activity in the neocortex of a head-fixed monkey through serial infusions of aCSF and muscimol, which respectively preserve and inhibit baseline unit firing activity, as depicted in FIG. 21A. The 1^{st} vertical line on left indicates the start of aCSF infusion (at 20 mins) and the 2^{nd} vertical line denotes the muscimol infusion (at 63.7 mins). The impedance measurement of the W-electrode was 1.5 MΩ in brain and at pre-implantation in saline was 2 MΩ. Modulation of neuronal firing activity was monitored by recording signals at the MiNDS electrode adjoining the infusion ports. The system was lowered until stable unit firing was observed to establish a baseline for comparison of firing rates and unit waveforms. Then, aCSF was infused for 5 mins 20 s at an infusion rate of 100 nl/min. This control infusion had minimal effect on the local firing rate and unit waveform during and after the infusion. Muscimol was then infused at the same location for 5 mins at infusion rate of 100 nl/min. This infusion immediately decreased the rate of detected spike activity, as depicted in FIG. 21B. As shown in FIG. 21B, the mean of the unit waveforms for each period following infusion are comparable and suggest that the same unit was being monitored throughout the serial infusion experiments, as depicted in FIG. 22. Vertical and horizontal bars denote 10 μV and 2 ms, respectively.

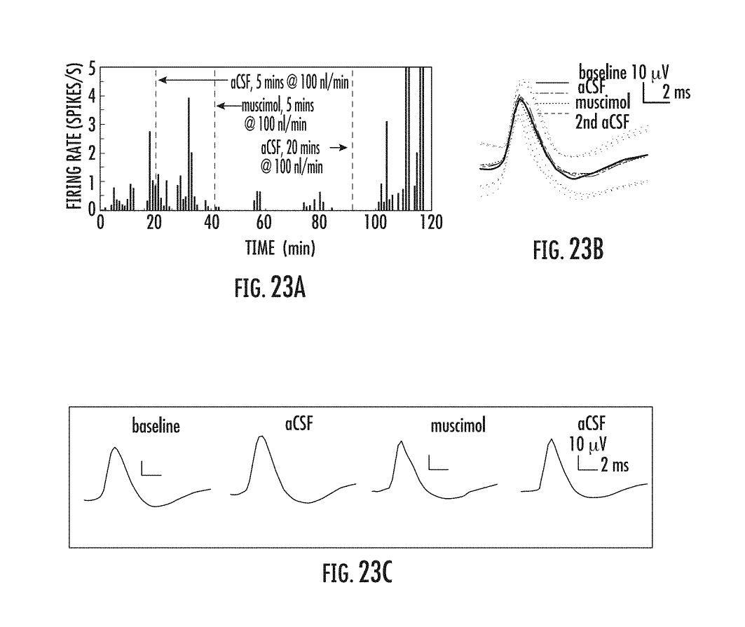

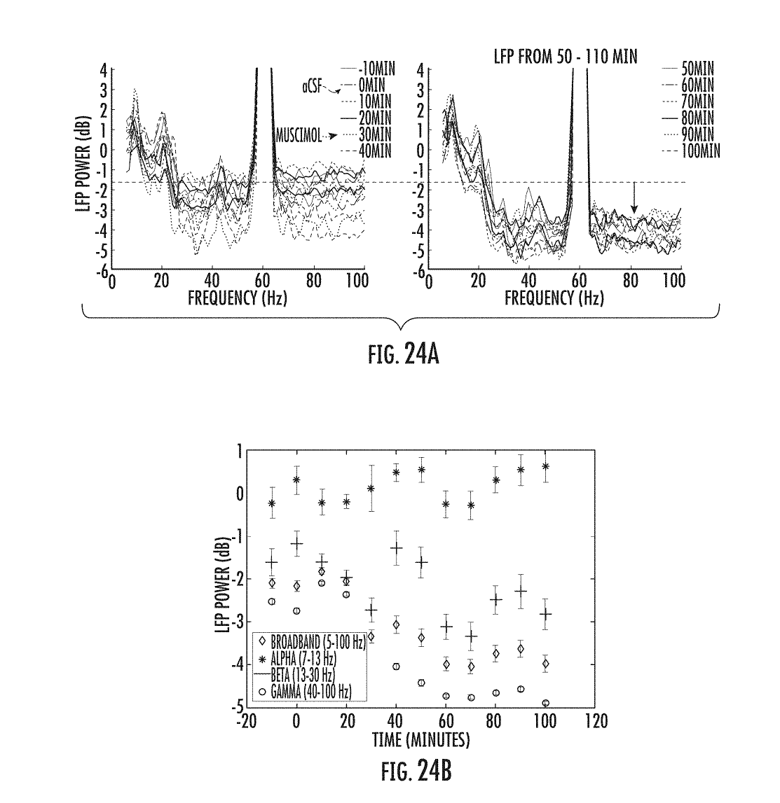

A second experiment was conducted targeting deeper layers of the neocortical region (i.e., dorsal bank of the cingulate cortex) to determine if recovery of the reduced neural activity through an additional infusion of aCSF could be induced at this adjoining site. This larger infusion of aCSF was indeed effective in reversing the inhibitory effect of muscimol, as shown by the observation of an increasing frequency of spikes with waveforms closely resembling those found during the pre-muscimol period, as depicted in FIGS. 23A-23C. In particular, the 1^{st} vertical line on left in FIG. 23A denotes the start of aCSF infusion (at 20 mins), the second vertical line represents the start of muscimol infusion (at 43 mins), and the third vertical line denotes the start of aCSF infusion (at 92 mins). In FIG. 23Cm vertical and horizontal bars denote 10 μV and 2 ms, respectively. This experiment demonstrated the fine, localized, bidirectional control capabilities of the system. Compared to prior work, this is the first system that allows serial infusion of multiple distinct solutions to be delivered in a focal, independent manner in a NHP. The system's integrated electrode can also be used to record local field potentials (LFPs), as shown in FIGS. 24A and 24B, which may be important for clinical applications that may require chronic recording from a fixed brain location. For example, pathological beta-band LFP in Parkinson's disease and/or epileptic discharges in epilepsy could be recorded from the chronically integrated electrode to track and treat dysfunction in future applications. In FIG. 24A, two curves for each period represent 95% confidence intervals. Each pair of curves correspond to signals averaged over 10 mins periods as labeled in the legend at the top right of each plot. It can be seen that broadband power from 30-100 Hz remains relatively consistent from baseline (−10 mins) to post aCSF infusion periods (0 min, 10 mins, 20 mins), and decreases immediately following muscimol infusion (30 mins, and all subsequent periods). Alpha (5-11 Hz) and beta (11-30 Hz) band power fluctuate without correlation to the infusions. The prominent power at 60 Hz is due to coupling of power mains noise. Relative baseline power is demarcated with a horizontal dashed black line across both plots to show relative changes in LFP broadband power that is especially visualized in the right plot, 20 minutes post-muscimol infusion (arrow). As can be seen in FIG. 24B, power decreases significantly for broadband, beta, and gamma frequency ranges, but persists for alpha frequencies.

Together, these experiments demonstrate that this minimally invasive, customizable device with high bending stiffness, high aspect ratio, adjustable number of channels (i.e., multi-functionality) in SS needle, can target the deep brain structures and can reliably modulate both local neural activity with cell-type specificity and behavior dependent upon this activity.

### Further Details of Example 5

Non-Invasive Brain Imaging Using Positron Emission Tomography (PET)

Radioactive Cu-64 was obtained from the Mallinckrodt Institute of Radiology (St. Louis, Mo.) in the form of Copper Chloride, and diluted with saline to 3 μCi/μl activity concentration. A Cu-64 solution was then infused intracerebrally into F344 Fischer Rats (Charles River Laboratories) using each of the following four methods:

(i) A 10 μl Luer lock syringe (#1701 Hamilton, Reno, Nev.) was connected to a 31G needle and pre-loaded with 5 ml of Cu-64 solution. A 1 mm burr hole was created in an untreated animal under isofluorane anesthesia 5 mm posterior to the bregma and 2 mm lateral from the midline (identical to MiNDS surgical procedure discussed above). The needle was lowered stereotaxically through the burr hole, 8 mm into the brain. 2 μl of Cu-64 was delivered using a Stoelting Quintessential Stereotaxic Injector, at a rate of 0.2 μl/min for 10 mins. The needle was left in place for 5 mins post end-infusion before being retracted slowly. The burr hole was then covered with bone wax and the cranial incision sutured with 5-0 non-resorbable monofilament suture. This protocol was used as an acute infusion case control, where the cannula was inserted only for the duration of the infusion and not chronically implanted.

(ii) Animals with an implanted MiNDS were anesthetized with isofluorane. One of the fluidic outputs of the device was connected to the same syringe/needle set up previously described in case (i). 1.67 μl of Cu-64 was delivered at a rate of 10 μl/hr for 10 mins. In another trial, 667 nl of Cu-64 was delivered at a rate of 10 μl/hr for 4 mins.

(iii) Animals with an implanted MiNDS were anesthetized with isofluorane. One of the fluidic outlets of the MiNDS was connected to an iPrecio pump. As in case (ii), one of two infusions were done: (1) 1.67 μl of Cu-64 was delivered at a rate of 10 μl/hr for 10 mins, or (2) 667 nl of Cu-64 was delivered at a rate of 10 μl/hr for 4 mins.

(iv) Agarose gel (0.6% by wt.) had a MiNDS implanted. This case is used as a control due to the similarity in mechanical properties to the brain tissue. As in case (ii), one of the fluidic outputs of the device was connected to the same syringe/needle set up previously described. One of two infusions was done: (1) 1.67 μl of Cu-64 was delivered at a rate of 10 μl/hr for 10 mins, or (2) 667 nl of Cu-64 was delivered at a rate of 10 μl/hr for 4 mins.

Immediately following the incision suture, in case (i) the anesthesized animal was imaged using a Perkin Elmer G8 PET /CT Preclinical Scanner for six 10 mins frames over the course of 30 mins. In cases (ii), (iii) & (iv) the anesthesized animal ((ii) & (iii)) or agarose phantom (iv) was imaged using a Perkin Elmer G8 PET /CT Preclinical Scanner for five 5 mins frames over the course of 20 mins. Imaging began prior to infusion, through infusion, and up to 5 mins post-end infusion. Images are reconstructed using MLEM 3D with 60 iterations. Intensity vs. position curves are shown in FIGS. 15, 16A-16C, and 25A-25C.

Only PET could be performed on the entire animal, due to the size of the bore and gantry. For case (i), CT was performed as well: the animal was euthanized using CO₂ asphyxiation and decapitated. The head was then imaged with PET and CT for a single 10 mins frame. Co-registration was then done with the original PET Data that was obtained in vivo and the PET/CT data that was obtained ex-vivo.

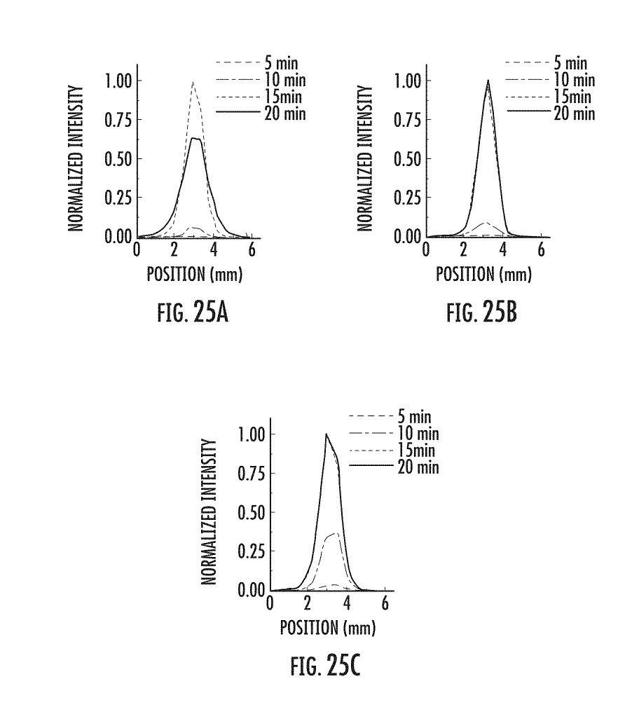

PET data was then analyzed in VivoQuant Analysis software (inviCRO, LLC, MA, USA) by using 2 methods: (1) by creating a 3D region of interest (ROI) around the infused bolus, and (2) by drawing a line profile horizontally across the maximum intensity plane of the bolus. The ROI was generated using connected thresholding techniques whereby the edges were defined by an intensity value equal to 10% the peak intensity at the center, I, for a total width, w. The summed intensity within the ROI was then calculated for each frame, and the results linearly normalized such that the maximum intensity value for each infusion case was equal to 1. The line profile analysis illustrated the diffusion behavior of the bolus over time in FIGS. 16A-16C and 25A-25C. Here, the total signal within ROI at a time point is the summed signal detected over the 5 mins exposure time of each scan.

Fabrication of the Tetrode Electrode

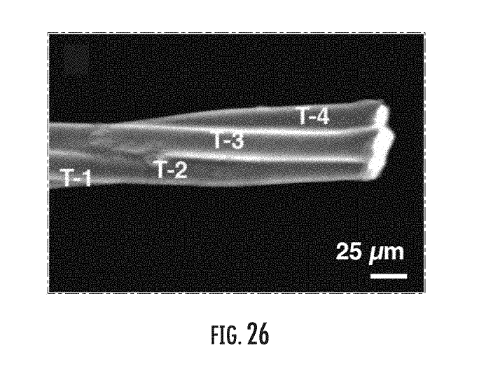

Each tetrode was built using two thin tungsten wires with a diameter of 20 μm and a length of 25 cm each. The wires were stuck together by running hands along them and then folded in half. The wires were hanged by the loop formed at one extremity and connected to a tetrode spinner (Neuralynx) from the other extremity. The four wires were twisted 130 turns forwards and then 15 turns backwards. Using a heat gun, the insulation of the tetrode was gently melted to increase its stiffness and the tightness of its tip, as depicted in FIG. 26.

Acute Recording for Tetrode MiNDS Study

As following the microfabrication steps depicted in FIGS. 2A-2J and 6A-6E, the W electrode was replaced with the tetrode W electrode, as depicted in FIG. 26. Adult female rats (F344) were anesthetized by exposure to isoflurane (2%, mixed with oxygen) and mounted in a stereotactic frame. A craniotomy was performed 2.5 mm posterior and 2.5 mm lateral to the bregma. A second craniotomy for the reference electrode was conducted 2.5 mm anterior to the bregma. A millmax pin was inserted into the brain to serve as the reference electrode. MiNDSs prepared with a tungsten tetrode as the electrode component were connected to an EIB board (Neuralynx, Bozeman, Mont.) which in turn interfaced to a PC via an Intan RHD 2000 USB interface board (Intan Technologies, Los Angeles, Calif.).

The dura was removed and the device was lowered into the brain to a depth of 2.5 mm and a location with unit activity was identified. The local neural signals were recorded with Open Ephys GUI software. Prior to drug infusion, the local signals were recorded for 30 mins to ensure stable spikes were located. After the baseline recording, 150 nl of saline were infused into the site at a flow rate of 100 nl/min and the activity was recorded for another 30 mins. Local silencing was achieved by the infusion of muscimol (1.0 mg/ml) via the other device channel (150 nl, 100 nl/min). Recording was recorded for another 30 mins post muscimol infusion. Saline washout was then performed by infusing 1.0 μl of saline at a flowrate of 100 nl/min and the activity was monitored until recovery, as depicted in FIG. 19.

MiNDS Implantation in Rats

F344 (SAS Fischer) rats were purchased from Charles River Laboratories and maintained under standard 12 hrs light/dark cycles. All materials used in surgeries were sterilized by autoclaving for 40 mins at 250° F. Rats were anesthetized with isofluorane before having their heads shaven and disinfected with alternating povidone-iodine (Betadine) and 70% ethanol scrubs, three times each. Animals underwent bilateral craniotomy and had a MiNDS implanted on the left side of the cortex and a ground screw implanted on the right hand side. The screw was placed such that the tip of the MiNDS did not penetrate the brain. Briefly, the animals were placed in a stereotactic frame, and a midline incision was made to expose the skull. Then, two burr holes were created using a dental drill. The left hand side burr hole was created using a 1 mm drill bit (Meisinger GmbH, Germany) and used for the MiNDS implantation, while the right hand side burr hole was made with a 0.5 mm drill bit and was used for the insertion of the ground reference screw. The ground screw was inserted 3 mm posterior to the bregma and 2 mm lateral to the midline, while the MiNDS was implanted approximately 5 mm posterior to the bregma and 2 mm lateral to the midline until reaching a depth of 8.5 mm, targeting the substantia nigra as described on the Paxinos and Watson Rat Brain atlas (6 ed.). The MiNDS and screw were then cemented to the skull using C&B Metabond adhesive (Parkell Inc., Edgewood N.Y.) and Orthojet dental cement (Lange Dental, Wheeling, Ill. USA), and the incision was closed using a 5-0 monofilament non-resorbable suture and 3M tissue glue. Custom made caps composed of 31G stainless steel connector coated with UV-cured epoxy were inserted into the protruding PEEK tubing to prevent dust and microbes from entering the tubing causing clogging and infection. Animals were ambulatory and healthy 1-week post-op.

Four animals were euthanized at 8 weeks post-surgery and used for biocompatibility studies. The rest were used for PET infusion studies outlined before.

High Pressure Liquid Chromatography (HPLC) Methods

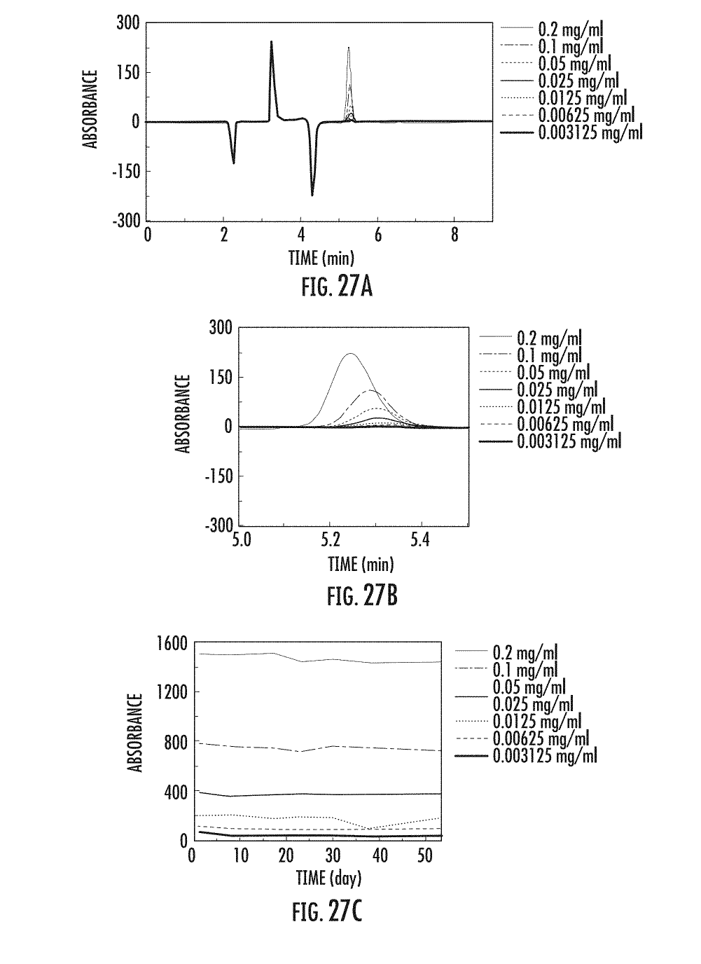

To ensure that constituted muscimol (used for the chronic behavioral studies) pre-loaded into the pumps would remain viable throughout the time implanted within the animal, a sample of the muscimol solution was stored in an incubator at 37° C. and HPLC assays were used to verify the stability of muscimol over 8 serial dilutions at 0.2, 0.1, 0.05, 0.025, 0.0125, 0.00625 and 0.003125 mg/ml (see FIGS. 27A-27C) at different time points for 2 months. This was done according to previously described protocols for HPLC characterization of muscimol. HPLC assays were performed on an Agilent 1200 series system with a 25 cm (L)×4.6 mm (ID) Spherisorb ODS-2 column (Waters, Millford, Mass., USA), containing 5 μm silica particles and 80 A pore size. The column was eluted with an aqueous solution of 0.5% v/v HBTA (heptafluorobutyric acid, Sigma Aldrich) at 1 ml/min. Then, 20 μl of samples were injected into the column and muscimol was detected using a UV detector with the absorbance wavelength set at 230 nm, and reference wavelength set at 360 nm. Muscimol concentration was determined by comparing the area under the curve at the appropriate retention time (5.5 mins) to a calibration curve of known concentrations.

Implantation of the MiNDS with the iPrecio Pumps in Rats

A similar protocol as described above was used to implant the MiNDS with the iPrecio pumps. Rats were anesthetized, and their heads were shaved and disinfected. A longer incision was made to accommodate the insertion of the pumps. As aforementioned, the MiNDS burr hole was made 5 mm posterior to the bregma and 2.5 mm lateral to the midline, and a support screw burr hole was made 3 mm posterior and 2 mm lateral to the bregma, on the opposite side. The screw was implanted, such that 1 mm protruded beneath the skull. A subcutaneous cavity for the pump was made by blunt dissection, and the pump was inserted through the incision. The MiNDS probe was implanted and cemented to the skull using C&B Metabond adhesive (Parkell Inc., Edgewood N.Y.) and Orthojet dental cement (Lange Dental, Wheeling, Ill. USA), and the incision was closed using 5-0 monofilament non-resorbable suture and 3M tissue glue. Animals were closely observed during the recovery period and given analgesics and wet food. By 1 week post-surgery, in all cases animals were ambulatory and otherwise healthy. Animals implanted with pumps were used for the behavioral studies described below. Pumps were pre-loaded and primed with either muscimol or saline. In all cases, animals displaying extensive post-operative morbidity more than 72 hrs post-surgery were euthanized and not further used in this study.

Behavioral Studies

A custom-made circular acrylic dish 1 foot in diameter and 3 feet in height was placed in an opaque black box. A GigE digital camera (resolution 750×480 pixels. The Imaging Source) was held in a stand such that it was directly above the dish, with the entire dish being in the field of view. The camera was connected to a computer where videos were acquired using IC Capture (The Imaging Source) and then imported into Ethovision software (Noldus) for further analysis.

The model presented here is based on a unilateral infusion of muscimol, a GABA agonist, and the subsequent measurement of contralateral and ipsilateral rotations done by the animal after infusion.

A rat was implanted with the MiNDS and two pumps pre-loaded and flushed with either saline or muscimol, as described above. The animal was placed within the dish and recorded over the course of 5 hrs. During the first 1 hr, no infusion was set. This was to establish a baseline recording of the animal's regular behavior. Then, Pump A infused 1.67 μl of saline for 10 mins. After 1 hr, pump B infused an identical 1.67 μl of muscimol (concentration 0.2 mg/ml) for 10 mins. The animal was further imaged for 3 hrs after the second infusion before being returned to its home cage. All experiments were done during the light hours of the animal's 12 hrs dark-light cycle.

Analysis for Behavioral Study

Videos were imported into Ethovision, where they were analyzed for ipsilateral and contralateral rotations over time. A rotation was defined as a 180° turn, as depicted in FIGS. 20A-20F, of the Center-Nose vector.

Selective Chemical Modulation in Awake Nonhuman Primate (NHP) Device and Infusion Preparation Procedures

Solutions used for infusion through the MiNDS were aCSF (artificial cerebrospinal fluid, Tocris Biosciences) and muscimol (2 mg/ml in aCSF, Sigma-Aldrich). The MiNDS and guide cannula were sonicated in a detergent solution (Alconox, Inc.), rinsed with water, and then sonicated and soaked in 70% ethanol followed by water until they were ready for implantation. Radel (Idex-HS) fluidic tubing and fittings were similarly cleaned. A Harvard 33 Twin Syringe Pump and microliter syringes (Model 702 RN SYR, Hamilton, Co.) were used for all infusion procedures. Prior to loading targeted solutions into the MiNDS, the cannulae were infused with 70% ethanol at 200 nl/min followed by water at 200 nl/min. The aCSF and muscimol solutions were individually backfilled into the two different tubing (prefilled with mineral oil) at a rate of 2 μl/min prior to being connected to the MiNDS ports. The targeted solutions were then infused through the MiNDS cannula at a rate of 100 nl/min for 30 mins to ensure sufficient permeation.

NHP Surgery

One rhesus monkey (6.5 kg female) (Macaca mulatta) was used. All experimental procedures were in accordance with the Institute Animal Care and Use Committee, followed guidelines of the MIT Committee on Animal Care, and complied with Public Health Service Policy on the humane care and use of laboratory animals. The monkey had been adapted to transitions from cages to a primate chair using pole-and-collar and food reinforcement. Experiments took place in a dark and electrically isolated chamber designed for NHP studies. The monkey had already been fitted with a chronic chamber and grid for electrode mapping. For the pilot studies reported here, the monkey received a chamber aimed at the cortex and the striatum in the right hemisphere and placed at an angle of 4° in the coronal plane for chronic recording under aseptic conditions and Sevofluorane anesthesia. Precise anatomical targeting was achieved by structural MRI (3 Tesla) to measure relative grid-hole coordinates. For the infusion and recording in primate, the MiNDSs were introduced into a chronically implanted guide tube or acutely introduced guide tube onto the chamber grid to target a structure and perform recording. Thus, the recording performed in the current study was not done during surgery but performed after surgery. Therefore, the MiNDS can be inserted into this system on any day to target specific brain regions without surgery.

Recording and Infusion Procedures for NHP Study

A micromanipulator (Narishige, MO-97A) was used to slowly lower the MiNDS after penetrating the dura matter using a 26 gauge guide tube (ConnHypo, 26G-XTW). The tip of the MiNDS was lowered into the cortex, or at targeted coordinates of anterior posterior, AP +23 mm (relative to interaural line) and mediolateral, ML +2 mm as estimated by grid holes that had been aligned to coronal MRI images.

Electrophysiological recording of spikes and local field potential was performed through Cheetah recording system (Neuralynx) using an HS-27 headstage. The reference and ground electrode were low-impedance (<1 kOhm) 75 μm diameter tungsten electrode placed inside the granulation tissue above the skull. Data were collected at a sampling rate of 32,556 samples/s with bandpass cut off frequencies at 0.5 Hz and 9000 Hz. For spike detection and sorting, data were high pass filtered at 300 Hz.

Once stable neuronal activity was confirmed based on consistent amplitudes of detected spikes, the MiNDS was secured at this position and a baseline recording was started for 30 mins. After baseline recording, 533 nl of aCSF was infused through one of the MiNDS cannulas followed by a 35 mins waiting period and subsequent 500 nl muscimol infusion through the second MiNDS cannula (both volumes infused at a rate of 100 nl/min). The aCSF infusion was shown to have minimal effect on local unit activity while muscimol immediately suspended activity as evidenced by the histograms shown in FIGS. 21A and 21B. This demonstrates the selective modulatory effects provided by the device's dual cannula system and the ability to resolve the modulated local neuronal activity.

Spike Sorting

Electrophysiological data was processed offline in Offline Sorter (Plexon) to identify single unit activity and in Neuroexplorer to create rate histograms based on these detected units. To identify individual units, the amplitude threshold for the highpass filtered data was set at 17 μV and sorted based on principle component analysis algorithms in Offline Sorter (T-Distribution Expectation Maximization) as well as user-input box templates to select expected ranges for peak and post-hyperpolarization waveforms. Waveforms for each period (baseline, aCSF infusion and post-infusion, muscimol infusion and post-infusion) were grouped to generate mean and standard deviations of the unit waveform over time, as depicted in FIGS. 21A-23C.

Data Analysis for Recorded Local Field Potential

As illustrated in FIGS. 24A and 24B, for analysis of recorded local field potentials in primate, recorded signals were first downsampled to an effective sampling rate of 1 kHz. All analyses were performed in Matlab Signals were analyzed in windowed periods of 700 ms with no overlap. Periods with large amplitude fluctuations (due to movement or other sources of noise) were removed by detecting signals greater than 0.15 mV in magnitude. Power spectra were generated by generating fast fourier transform averaged over clean 700 ms periods of LFP squared (i.e., power) from a 10 mins interval to display changes in gross LFP power over the course of infusions and between these infusions. Spectra were computed through a multitaper method using a single taper with a time bandwidth product of 1.8. Significance boundaries were set using a p level of 0.05 and these boundaries are displayed by the two curves generated for each spectrum for each 10 mins time period. A pink noise spectrum was log-fit to the baseline LFP power (first 10 minute of recording before any infusion) using p=afb, where f indicates frequency, and the parameters a and b were fitted to the power averaged over this period. This pink-fit power was then removed from all generated spectra to improve visualization of the relevant changes at a broad range of frequencies. Power fluctuations across the 10 mins periods are displayed as relative changes in log-scale (dB) to the baseline (‘−10 mins’ labeled curve).

Although specific embodiments of the disclosure have been described, numerous other modifications and alternative embodiments are within the scope of the disclosure. For example, any of the functionality described with respect to a particular device or component may be performed by another device or component. Further, while specific device characteristics have been described, embodiments of the disclosure may relate to numerous other device characteristics. Further, although embodiments have been described in language specific to structural features and/or methodological acts, it is to be understood that the disclosure is not necessarily limited to the specific features or acts described. Rather, the specific features and acts are disclosed as illustrative forms of implementing the embodiments.

We claim:

1. A drug delivery system, comprising:
two or more discrete, annular microtubes, wherein each of the two or more microtubes comprises a distal end, a proximal end, and elongate channel body extending therebetween;
an electrode comprising a distal end, a proximal end, and elongate body extending therebetween;
an elongate carrying template supporting the two or more microtubes and the electrode in an aligned stack;
an annular needle having distal end and a proximal end, an annulus of the needle housing the carrying template, the two or more microtubes, and the electrode; and
at least one pump fluidically connected to the proximal end(s) of one or more of the two or more microtubes, wherein the at least one pump is configured to deliver a fluid drug on demand through the elongate channel body and out of the distal end of the one or more microtubes.

2. The drug delivery system of claim 1, wherein each of the microtubes has an outer diameter of about 30 microns and an inner diameter of about 20 microns.

3. The drug delivery system of claim 1, wherein the elongate carrying template comprises a microfabricated polyimide structure.

4. The drug delivery system of claim 1, wherein the annular needle comprises a stainless steel.

5. The drug delivery system of claim 1, wherein the annular needle has an outer diameter of about 200 microns.

6. The drug delivery system of claim 1, wherein the annular needle has an aspect ratio (length:diameter) of at least 500.

7. The drug delivery system of claim 1, wherein the electrode is a tungsten electrode.

8. The drug delivery system of claim 1, wherein the electrode comprises an electrically insulating oxide coating between its proximal and distal ends.

9. The drug delivery system of claim 1, wherein the annular needle, the electrode, and the two or more microtubes have a length from about 1 cm to about 10 cm.

10. The drug delivery system of claim 1, wherein the microtubes are formed of borosilicate.

11. The drug delivery system of claim 1, further comprising an aligner tip securing the distal ends of the electrode and the two or more microtubes at a fixed position about the distal end of the annular needle.

12. The drug delivery system of claim 11, wherein the aligner tip is formed of borosilicate.

13. The drug delivery system of claim 1, which is configured for delivery of the fluid drug to a neural tissue site.

14. A method for local delivery of a fluid drug into a patient in need thereof, comprising:
inserting the distal ends of the annular needle, the electrode, and the two or more microtubes of the drug delivery system of claim 1 into a selected target tissue site in the patient; and
delivering one or more doses of the fluid drug to the selected tissue site via at least one of the microtubes of the drug delivery system.

15. The method of claim 14, wherein selected target tissue site is a neural network site in the patient's brain.

16. The method of claim 14, wherein each of the one or more doses of the fluid drug is a bolus from about 17 nL to about 2 μL.

17. The method of claim 14, wherein the fluid drug comprises a neuromodulating agent.

18. The method of claim 17, wherein the neuromodulating agent comprises muscimol or another GABA agonist.

19. The method of claim 14, wherein selected target tissue site is a neural network site in the patient's brain, wherein each of the one or more doses of the fluid drug is a bolus from about 17 nL to about 2 μL, and wherein the fluid drug comprises a neuromodulating agent.

20. A method for neural circuit modulation in a patient in need thereof, comprising:
inserting the distal ends of the annular needle, the electrode, and the two or more microtubes of the drug delivery system of claim 1 into a neural network site in the patient; and
delivering one or more doses of the fluid drug to the neural network site via at least one of the microtubes of the drug delivery system.

21. The method of claim 20, wherein the neural network site is in the patient's brain.

22. The method of claim 20, wherein the neural network site is in a deep brain structure.

23. The method of claim 20, wherein each of the one or more doses of the fluid drug is a bolus from about 17 nL to about 2 μL.

24. The method of claim 20, wherein the fluid drug comprises a neuromodulating agent.

25. The method of claim 24, wherein the neuromodulating agent comprises muscimol or another GABA agonist.

26. A neuromodulation system, comprising:
two or more discrete, annular microtubes, wherein each of the two or more microtubes comprises a distal end, a proximal end, and elongate channel body extending therebetween;
an electrode comprising a distal end, a proximal end, and elongate body extending therebetween;
an elongate carrying template supporting the two or more microtubes and the electrode in an aligned stack;
an annular needle having distal end and a proximal end, an annulus of the needle housing the carrying template, the two or more microtubes, and the electrode; and
at least one neuromodulator source operatively coupled to at least one of the two or more microtubes, wherein the neuromodulator source is selected from:
a pump fluidically connected to the proximal end of one of the two or more microtubes and configured to deliver a fluid drug on demand through the distal end of at least one of the microtubes; and
an optical fiber configured to deliver light out from the distal end of at least one of the microtubes.

27. A method of assembly of a drug delivery system, the method comprising:
providing two or more discrete, annular microtubes, wherein each of the two or more microtubes comprises a distal end, a proximal end, and elongate channel body extending therebetween;
providing an electrode comprising a distal end, a proximal end, and elongate body extending therebetween;
supporting, by an elongate carrying template, the two or more microtubes and the electrode in an aligned stack; and
housing, at least partially within an annulus of an annular needle having distal end and a proximal end, the carrying template, the two or more microtubes, and the electrode.

28. The method of claim 27, further comprising fluidically connecting at least one pump to the proximal end(s) of one or more of the two or more microtubes, wherein the at least one pump is configured to deliver a fluid drug on demand through the elongate channel body and out of the distal end of the one or more microtubes.
